(19) United States
(12) Patent Application Publication
(10) Pub. No.: US 2025/0347694 A1
(43) Pub. Date: Nov. 13, 2025

(54) BIOLOGICAL MARKERS OF LIVER FAT

(71) Applicants: John O'Sullivan, New South Wales (AU); Roman Rodionov, Dresden (DE); Stephanie Bode Boger, Magdeburg (DE); Jens Martens Lobenhoffer, Magdeburg (DE)

(72) Inventors: John O'Sullivan, New South Wales (AU); Roman Rodionov, Dresden (DE); Stephanie Bode Boger, Magdeburg (DE); Jens Martens Lobenhoffer, Magdeburg (DE)

(21) Appl. No.: 18/690,465

(22) PCT Filed: Sep. 12, 2022
(86) PCT No.: PCT/AU2022/051103

§ 371 Date: Mar. 8, 2024

(30) Foreign Application Priority Data
Sep. 10, 2021 (AU) 2021902940

(51) Int. Cl. G01N 33/574; G01N 24/08; G01N 30/72; G01N 33/573; G01N 33/68; G01N 33/72; G01N 33/86

(52) U.S. Cl. CPC G01N 33/57438; G01N 33/573; G01N 33/6893; G01N 33/728; G01N 33/86; G01N 24/08; G01N 30/7206; G01N 30/7233; G01N 2800/085

(57) ABSTRACT

The present invention provides diagnostic and/or prognostic markers indicative of liver fat, fatty liver diseases (e.g. non-alcoholic fatty liver disease (NAFLD) and/or hepatocellular cancer deriving from liver fat.

### INCORPORATION BY CROSS-REFERENCE

This application claims priority to International Application No. PCT/AU2022/051103, filed on Sep. 12, 2022, which claims priority to Australia Application No. 2021902940, filed on Sep. 10, 2021, each of which is incorporated herein in its entirety for all purposes.

### TECHNICAL FIELD

The present invention relates generally to the field of biological markers, and in particular diagnostic and/or prognostic markers indicative of liver fat and hepatocellular cancer deriving from liver fat. The invention provides methods for the detection of markers of liver fat and liver-fat induced liver cancer. Also provided are methods for the diagnosis and/or prognosis of fatty liver diseases, non-limiting examples of which include non-alcoholic fatty liver disease (also known as metabolic associated fatty liver disease) and alcoholic fatty liver disease. Also provided are methods for the diagnosis of hepatocellular cancer deriving, for example, from non-alcoholic fatty liver disease.

### BACKGROUND

Fatty liver disease (FLD), also known as hepatosteatosis, is a prevalent liver condition which occurs when lipids accumulate in liver cells. The accumulated lipids can cause cellular injury and sensitise the liver to further injury. They can also impair hepatic microvascular circulation.

FLD may arise from a number of sources, including excessive consumption of alcohol and metabolic disorders, including those associated with obesity, insulin resistance, and hypertension. Non-alcoholic fatty liver disease (NAFLD) can also result from metabolic disorders including, for example, homocystinuria, galactosemia, tyrosemia, and glycogen storage diseases, as well as dietary conditions such as malnutrition, total parenteral nutrition, starvation, and overnutrition. In some cases, NAFLD is linked with jejunal bypass surgery, and other causes include specific medications, including amiodarone, maleate, corticosteroids, methotrexate, estrogens (e.g. synthetic estrogens), perhexilin, tamoxifen, and nucleoside analogs, as well as exposure to certain chemicals including hydrocarbon solvents. Acute fatty liver conditions can also arise during pregnancy.

FLD can progress to more advanced liver disease such as nonalcoholic steatohepatitis (NASH), also known as metabolic steatohepatitis. NASH is characterised by liver damage and inflammation and can be accompanied by cirrhosis or fibrosis of the liver. NASH may culminate in further liver damage and chronic liver failure, in some cases with hepatocellular carcinoma.

The prevalence of FLD, and its significantly adverse health outcomes, make it a key health issue worldwide.

As noted above, non-alcoholic fatty liver disease (NAFLD), more recently termed “metabolic associated fatty liver disease” or MAFLD¹⁻³, is a prominent form of FLD. It is the most common liver disease worldwide⁴ and has primarily been driven by the modem pandemic of overweight/obesity. In the USA, one third of the adult population and 10-20% of children suffer from NAFLD. The prevalence in Europe and Asia is slightly lower, but still affects about 25% of adults.⁵ Risk factors for NAFLD include, but are not limited to, central obesity, type 2 diabetes, and dyslipidemia.⁶ In 20-30% of cases, NAFLD progresses to steatohepatitis (metabolic steatohepatitis), which can progress to fibrotic liver disease known as cirrhosis, or to liver cancer.

Alanine aminotransferase (ALT) is the most sensitive blood test currently available for MAFLD, followed by aspartate aminotransferase (AST)⁷, however their ability to track change in NAFLD over time is unknown. Fatty liver index (FLI), developed by Bedogni et al. in 2006,¹⁹ is an algorithm that can be used in the clinic to predict fatty change in the liver. It includes body mass index, waist circumference, triglyceride and gamma glutamyltransferase levels.⁸ Most patients diagnosed with NAFLD do not have any symptoms, with some complaining of mild upper quadrant pain related to fatty infiltration of the liver and capsular distension. There are no specific signs or symptoms related to the early stages of MAFLD, and ultrasound has low sensitivity when liver fat content is less than 20%.⁹ Clearly, noninvasive, cheap, sensitive, and easily-accessible diagnosis of NAFLD with ability to track change over time would facilitate screening and monitoring of patients at risk, as well as renew enthusiasm for clinical trials looking at novel therapeutic options.

Fatty liver disease has exploded in prevalence, but its accurate detection remains a diagnostic challenge, as does its tracking over time. The current standard of practice is initial detection by plasma alanine or aspartate aminotransferase (ALT, AST) if clinically suspicious, and if these are abnormal then referral for targeted liver imaging. Currently, a highly accurate modality for quantifying liver fat is magnetic resonance imaging (MRI) fat-free weight (FFW). More recently, MR spectroscopy has been used to determine liver fat down to the 5% level, reported to be the true level of normal versus abnormal levels of liver fat.

There is an unmet need for improved diagnostic tests for liver fat detection and/or the accurate detection of changes in liver fat over time. There is also an unmet need for improved methods of diagnosing and/or prognosing hepatocellular cancer.

### SUMMARY OF THE INVENTION

The present invention alleviates at least one of the problems in the prior art in providing improved markers for liver fat detection. As demonstrated herein, dimethylguanidino valeric acid, in both its symmetric (referred to herein as “SDGV”) and asymmetric (referred to herein as “ADGV”) isomers provides superior diagnostic outcomes in fatty liver detection over ALT and AST. In particular, the present inventors have identified that SDGV has superior capacity to rule in and rule out liver fat compared to ALT, AST and ADGV.

Furthermore, it is demonstrated herein that both SDGV and ADGV have superior tracking capability of liver fat over time, as compared to ALT and AST. In particular, SDGV, and not ADGV, is demonstrated to be a faithful reporter of activity of enzyme alanine glyoxylate aminotransferase 2 (AGXT2), which has a causal role in non-alcoholic fatty liver disease (NAFLD).

Also described herein are improved methods for diagnosing and/or prognosing hepatocellular carcinoma (HCC) in subjects. The present inventors have identified that the biological marker taurodeoxycholic acid (TDCA) is a reliable indicator of hepatocellular carcinoma, particularly in subjects that have been determined to be suffering from fatty liver diseases (e.g. NAFLD).

Accordingly, the present invention provides improved diagnostic and prognostic markers for liver fat and fatty liver disease and/or hepatocellular carcinoma which, in comparison to existing methods, may also be more accurate, more cost-effective, more efficient, and/or less invasive.

In a first aspect, the present invention provides a method for detecting liver fat in a subject, the method comprising measuring the level of symmetric dimethylguanidino valeric acid (SDGV) in a biological sample obtained from the subject, and determining whether the subject has liver fat based upon the level of SDGV in the body fluid sample.

In a second aspect, the present invention provides a method for determining a presence or absence of liver fat in a subject, the method comprising measuring the level of symmetric dimethylguanidino valeric acid (SDGV) in a biological sample obtained from the subject, and determining said presence or absence of liver fat based on the level of SDGV in the body fluid sample.

In a third aspect, the present invention provides a method for determining a subject's level of liver fat, the method comprising measuring the level of symmetric dimethylguanidino valeric acid (SDGV) in a biological sample obtained from the subject, and determining the level of liver fat based on the level of SDGV in the body fluid sample.

In a fourth aspect, the present invention provides a method for determining a change in liver fat level in a subject, the method comprising measuring the level of symmetric dimethylguanidino valeric acid (SDGV) in biological samples obtained from the subject at multiple timepoints, and determining whether there is a change in liver fat level in the subject over time based on a comparison of the level of SDGV in each said body fluid sample.

In an embodiment of the first to fourth aspects, the subject has a fatty liver disease (FLD).

In a fifth aspect, the present invention provides a method for diagnosing and/or prognosing fatty liver disease (FLD) in a subject, the method comprising measuring the level of symmetric dimethylguanidino valeric acid (SDGV) in a biological sample obtained from the subject, and determining whether the subject has fatty liver disease based on the level of SDGV in the body fluid sample.

In an embodiment of the first to fifth aspects, the fatty liver disease (FLD) is non-alcoholic fatty liver disease (NAFLD).

In another embodiment of the first to fifth aspects, the fatty liver disease (FLD) is alcoholic liver disease (ALD).

In one embodiment of the first to fifth aspects, measuring the level of symmetric dimethylguanidino valeric acid (SDGV) in the biological sample is performed without measuring the level of asymmetric dimethylguanidino valeric acid (ADGV) in the biological sample.

In another embodiment of the first to fifth aspects, said determining based upon the level of SDGV in the body fluid sample is performed without determining based on the level of asymmetric dimethylguanidino valeric acid (ADGV) in the biological sample.

In an additional embodiment of the first to fifth aspects, the level of symmetric dimethylguanidino valeric acid (SDGV) in the biological sample is compared to that of a SDGV control.

The SDGV control may be selected from any one or more of:
a biological sample from a subject without liver fat, or without clinically acceptable levels of liver fat;
a series of biological samples from subjects without liver fat or without clinically acceptable levels of liver fat;
a standard control value or a set of standard control values indicative of the presence or absence of liver fat.

The clinically acceptable levels of liver fat may each be: less than 20% fat content in liver by weight; less than 15% fat content in liver by weight; less than 10% fat content in liver by weight; less than 8% fat content in liver by weight; less than 7% fat content in liver by weight; less than 6% fat content in liver by weight; less than 5% fat content in liver by weight; or less than 4% fat content in liver by weight.

The standard control value or a set of standard control values indicative of the presence of liver fat may be each be: more than 4% fat content in liver by weight; more than 5% fat content in liver by weight; more than 6% fat content in liver by weight; more than 7% fat content in liver by weight; more than 8% fat content in liver by weight; more than 10% fat content in liver by weight; more than 15% fat content in liver by weight; or more than 20% fat content in liver by weight.

The standard control value or a set of standard control values indicative of the absence of liver fat may each be: less than 20% fat content in liver by weight; less than 15% fat content in liver by weight; less than 10% fat content in liver by weight; less than 8% fat content in liver by weight; less than 7% fat content in liver by weight; less than 6% fat content in liver by weight; less than 5% fat content in liver by weight; or less than 4% fat content in liver by weight.

In another embodiment of the first to fifth aspects, said measuring further comprises measuring the level of a further biological marker in the biological sample selected from any one or more of: alanine transaminase (ALT), aspartate transaminase (AST), alkaline phosphatase (ALP), albumin, albumin and total protein, bilirubin, gamma-glutamyltransferase (GGT), L-lactate dehydrogenase (LD), prothrombin time (PT) and/or taurodeoxycholic acid (TDCA); and
said determining is additionally based on the level of the further biological marker in the biological sample.

The level of the further biological marker in the biological sample may be compared to that of a further control.

In an additional embodiment of the first to fifth aspects, the biological sample is a blood, serum, plasma, urine or liver sample.

In one embodiment of the first to fifth aspects, the method further comprises culturing the biological sample prior to said determining.

In another embodiment of the first to fifth aspects, the method comprises the step of obtaining the biological sample from the subject.

In still another embodiment of the first to fifth aspects, the subject is a mammal, a non-human mammal, murine, a primate, or a human.

In a further embodiment of the first to fifth aspects, the measuring of the level of symmetric dimethylguanidino valeric acid (SDGV) in the biological sample is conducted by any one or more of tandem liquid chromatography-mass spectrometry (LC-MS/MS), gas chromatography-mass spectrometry (GC-MS/MS), and/or nuclear magnetic resonance spectroscopy (NMR).

In a sixth aspect, the present invention provides a method for diagnosing and/or prognosing hepatocellular carcinoma (HCC) in a subject, the method comprising:
determining if the subject has non-alcoholic fatty liver disease (NAFLD); and
determining whether the subject has HCC by measuring the level of taurodeoxycholic acid (TDCA) in a biological sample obtained from the subject, and determining whether the subject has HCC based on the level of TDCA in the body fluid sample.

In one embodiment of the sixth aspect, said determining if the subject has non-alcoholic fatty liver disease (NAFLD) is by measuring the level of symmetric dimethylguanidino valeric acid (SDGV) in a biological sample obtained from the subject, and determining whether the subject has fatty liver disease based on the level of SDGV in the body fluid sample.

In one embodiment of the sixth aspect, said determining if the subject has non-alcoholic fatty liver disease (NAFLD) comprises or consists of performing the method of any one of the first to fifth aspects.

In another embodiment of the sixth aspect,
the level of symmetric dimethylguanidino valeric acid (SDGV) in the biological sample is compared to that of a SDGV control; and/or
the level of the TDCA in the biological sample is compared to that of a TDCA control.

In another embodiment of the sixth aspect, the SDGV control and/or the TDCA control is selected from any one or more of:
a biological sample from a subject without liver fat and without HCC, or without clinically acceptable levels of liver fat and without HCC;
a series of biological samples from subjects without liver fat and without HCC, or without clinically acceptable levels of liver fat and without HCC;
a standard control value or a set of standard control values indicative of the presence or absence of liver fat and/or HCC.

In still another embodiment of the sixth aspect, said determining if the subject has non-alcoholic fatty liver disease (NAFLD) comprises determining a concentration of the SDGV in plasma from the subject of above 0.4 μM, above 0.45 μM, above 0.5 μM, above 0.51 μM, above 0.516 μM, above 0.52 μM or above 0.525 μM, to thereby determine that the subject has NAFLD.

In another embodiment of the sixth aspect, said determining whether the subject has HCC comprises determining a concentration of the TDCA in a plasma from the subject of above, above 0.7 μM, above 0.75 μM, or above 0.8 μM, to thereby determine that the subject has HCC.

In another embodiment of the sixth aspect, the method comprises:
determining a concentration of the SDGV in plasma from the subject of below 0.525 μM, below 0.52 μM, below 0.516 μM, below 0.51 μM, below 0.5 μM, below 0.45 μM or below 0.4 μM; and/or
determining a concentration of the TDCA in a plasma from the subject of below 0.8 μM, below 0.75 μM, or below 0.7 μM;
to thereby determine that the subject does not have HCC.

In one embodiment of the sixth aspect, the method comprises:
determining a concentration of the SDGV in plasma from the subject of above 0.4 μM, above 0.45 μM, above 0.5 μM, above 0.51 μM, above 0.516 μM, above 0.52 μM or above 0.525 μM, to thereby determine that the subject has NAFLD; and
determining a concentration of the TDCA in a plasma from the subject of below 0.8 μM, below 0.75 μM, or below 0.7 μM;
to thereby determine that the subject should be monitored due to a risk of developing HCC.

In one embodiment of the sixth aspect, the subject is monitored by periodic measurement of SDGV levels in plasma from the subject and/or periodic measurement of TDCA levels in plasma from the subject.

In another embodiment of the sixth aspect, the method comprises the step of obtaining the biological sample from the subject.

In another embodiment of the sixth aspect, the subject is a mammal, a non-human mammal, murine, a primate, or a human.

In a further embodiment of the first to sixth aspects, the method further comprises either of:
(i) treating the subject to reduce liver fat in the subject;
(ii) treating the subject to alleviate a disease arising at least in part from or characterised by excess liver fat.

In one embodiment, the disease arising at least in part from or characterised by excess liver fat is hepatocellular carcinoma (HCC).

In a seventh aspect, the present invention provides use of an agent capable of specifically detecting symmetric dimethylguanidino valeric acid (SDGV) in a biological sample in the preparation of a medicament for:
detecting liver fat in a subject;
determining a presence or absence of liver fat in a subject;
determining a subject's level of liver fat;
determining a change in liver fat level in a subject;
diagnosing and/or prognosing fatty liver disease (FLD) in a subject; and/or diagnosing and/or prognosing non-alcoholic fatty liver disease (NAFLD) in a subject.

In an eighth aspect, the present invention provides an agent capable of specifically detecting symmetric dimethylguanidino valeric acid (SDGV) in a biological sample for use in:
detecting liver fat in a subject;
determining a presence or absence of liver fat in a subject;
determining a subject's level of liver fat;
determining a change in liver fat level in a subject;
diagnosing and/or prognosing fatty liver disease (FLD) in a subject; and/or
diagnosing and/or prognosing non-alcoholic fatty liver disease (NAFLD) in a subject.

In a ninth aspect, the present invention provides use of an agent capable of specifically detecting symmetric dimethylguanidino valeric acid (SDGV) in a biological sample, and an agent capable of specifically detecting taurodeoxycholic acid (TDCA) in a biological sample, in the preparation of a medicament or a combination of medicaments for diagnosing and/or prognosing hepatocellular carcinoma (HCC) in a subject.

In a tenth aspect, the present invention provides an agent capable of specifically detecting symmetric dimethylguanidino valeric acid (SDGV) in a biological sample, and an agent capable of specifically detecting taurodeoxycholic acid (TDCA) in a biological sample, for use in diagnosing and/or prognosing hepatocellular carcinoma (HCC) in a subject, wherein each said agent is used in combination either sequentially or simultaneously.

In an eleventh aspect, the present invention provided a kit for use in, or when used for, the method of the first to fifth aspects, the kit comprising any one or more of: an agent capable of specifically detecting symmetric dimethylguanidino valeric acid (SDGV) in a biological sample;
an agent capable of specifically detecting taurodeoxycholic acid (TDCA) in a biological sample;
instruments and/or reagents for obtaining and/or processing the sample;
wash reagents, buffers and/or enzymes used in performing the method;
written instructions for use of the kit in the method.

In a twelfth aspect, the present invention provides use of a kit in the method of any of the first to fifth aspects, wherein the kit comprises any one or more of:
an agent capable of specifically detecting symmetric dimethylguanidino valeric acid (SDGV) in a biological sample;
an agent capable of specifically detecting taurodeoxycholic acid (TDCA) in a biological sample;
instruments and/or reagents for obtaining and/or processing the sample;
wash reagents, buffers and/or enzymes used in performing the method;
written instructions for use of the kit in the method.

### Definitions

As used in this application, the singular form “a”, “an” and “the” include plural references unless the context clearly dictates otherwise. For example, the term “cell” also includes multiple cells unless otherwise stated.

As used herein, the term “comprising” means “including”, in a non-exhaustive sense. Variations of the word “comprising”, such as “comprise” and “comprises” have correspondingly varied meanings. Thus, for example, a biological sample “comprising” a given component A may consist exclusively of component A, or may include one or more additional components such as component B.

As used herein, the term “metabolic associated fatty liver disease” and “MAFLD” will be understood to have an equivalent meaning to “non-alcoholic fatty liver disease” or “NAFLD”.

As used herein, the terms “term fatty liver disease” and “FLD”, refer to a pathological condition or disease caused at least in part by abnormal hepatic lipid deposits. Fatty liver disease/FLD includes, for example, alcoholic fatty liver disease, nonalcoholic fatty liver disease, and acute fatty liver of pregnancy. Fatty liver disease/FLD may include, for example microvesicular steatosis and macrovesicular steatosis. In some embodiments, fatty liver disease/FLD is characterised by a lipid (e.g. triglyceride) content in the liver of above: 5%, 6%, 7%, 8%, 10%, 15%, or 20% by weight as measured, for example, by standard imaging or biopsy techniques, and/or magnetic resonance spectroscopy using ¹H-MRS⁸.

As used herein “fatty liver” and “liver fat” will be understood to be a reference to accumulated lipid (e.g. triglycerides) in the liver.

As used herein the terms “SDGV” and “symmetric dimethylguanidino valeric acid” will be understood to mean an isomer of dimethylguanidino valeric acid having one methyl group on the primary amine nitrogen and one methyl group on the imine nitrogen, as depicted below:

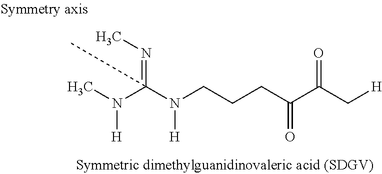

As used herein the terms “ADGV” and “asymmetric dimethylguanidino valeric acid” will be understood to mean an isomer of dimethylguanidino valeric acid having two/both methyl groups on the primary amine nitrogen, as depicted below:

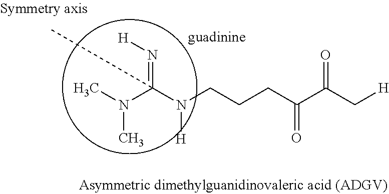

As used herein, the term “between” when used in reference to a range of numerical values encompasses the numerical values at each endpoint of the range.

As used herein, the term “about”, when used in reference to a recited numerical value, includes the recited numerical value and numerical values within plus or minus ten percent of the recited value.

As used herein, the terms “treat”, “treating”, “treatment”, and the like refer to reducing or ameliorating a disorder/disease and/or symptoms associated therewith. It will be appreciated, although not precluded, that treating a disease, disorder or condition does not require that the disease, disorder, condition, or symptoms associated therewith be completely eliminated.

As used herein, the term “subject” includes any animal of economic, social or research importance including bovine, equine, ovine, primate, avian and rodent species. Hence, a “subject” may be a mammal such as, for example, a human or a non-human mammal.

### BRIEF DESCRIPTION OF THE FIGURES

Preferred embodiments of the present invention will now be described by way of example only, with reference to the accompanying figures wherein:

FIG. 1 shows A. changes in liver fat (y-axis) correlated with changes in BMI in the PLIS cohort. Spearman correlation r=0.444; and B. Baseline liver fat correlated to change in liver fat. FFW: Fat-Free Weight=liver fat quantification by MRI. BMI: body mass index;

FIG. 2 shows change in biomarker vs change in liver fat: SDGV A. and ALT B. FFW: fat-free weight determined by liver MRI; SDGV_lnA: change in symmetric dimethylguanidino valeric acid, log-scale; ALT_lnA: change in alanine aminotransferase, log-scale; r=correlation coefficient; P=p-value;

FIG. 3 provides representative images from hematoxylin-eosin stained liver slides from A. db/+ and B. db/db mice. White spaces (arrows) indicate accumulation of hepatic fat. Magnification ×200;

FIG. 4 shows A. protein level and B. specific activity, of alanine:glyoxylate aminotransferase 2 (AGXT2) in livers and kidneys of db/db and db/+ mice. D6-ADGV: stable-isotope labelled α-keto-dimethylguanidinovaleric acid;

FIG. 5 shows plasma levels of alanine:glyoxylate aminotransferase 2 substrates (A. SDMA, B. ADMA, C. homoarginine) and products (D. SDGV, E. ADGV, F. GOCA) in db/db and db/+ mice. ADMA: asymmetric dimethylarginine; SDMA: symmetric dimethylarginine; GOCA: 6-guanidino-2-oxocaproic acid; ADGV: asymmetric α-keto-dimethylguanidinovaleric acid; SDGV: symmetric α-keto-dimethylguanidinovaleric acid;

FIG. 6 shows representative ion chromatograms on a hypercarb column. A. separate ADGV and SDGV ion chromatograms when injecting into LCMS individually. B. total ion chromatograms of ADGV, SDGV and their respective deuterated forms. C. Deuterated TDCA and native TDCA were eluted later around 14 min on the hypercarb column;

FIG. 7 shows standard curves of ADGV, SDGV (7A, 7B), and TDCA (7C). Deuterated ADGV, SDGV (5 ng/mL), TDCA (10 ng/mL) used as internal standards were added into all concentrations of ADGV, SDGV and TDCA solutions before LC-MS/MS analysis;

FIG. 8 shows the results of an analysis of SDGV and TDCA quantifications in plasma samples of a patient cohort;

FIG. 9 provides A. a graph demonstrating that taurodeoxycholate (TDCA) is able to distinguish between hepatocellular carcinoma (HCC) subjects and non-HCC subjects in a cohort of subjects with non-alcoholic fatty liver disease (NAFLD), and B. a receiver operating characteristic (ROC) curve analysis of TDCA for the HCC versus non-HCC subjects; and

FIG. 10 provides A. a graph demonstrating that taurodeoxycholate (TDCA) is able to distinguish between hepatocellular carcinoma (HCC) subjects and non-HCC subjects in a cohort of subjects with non-alcoholic fatty liver disease (NAFLD), and B. a receiver operating characteristic (ROC) curve analysis of TDCA for the HCC versus non-HCC subjects.

### DETAILED DESCRIPTION

The following detailed description conveys exemplary embodiments of the present invention in sufficient detail to enable those of ordinary skill in the art to practice the present invention. Features or limitations of the various embodiments described do not necessarily limit other embodiments of the present invention, or the present invention as a whole. Hence, the following detailed description does not limit the scope of the present invention, which is defined only by the claims.

The present invention provides improved markers for detecting the presence or absence of liver fat, and/or for tracking changes in liver fat over time. In particular, the new markers described herein are demonstrated to provide superior results compared to existing approaches based on alanine aminotransferase (ALT) and aspartate aminotransferase (AST). It has been determined by the present inventors that the diagnostic performance of dimethylguanidino valeric acid, in both its individual symmetric (SDGV) and asymmetric (ADGV) isomers, exceeds that of ALT and AST. Furthermore, both SDGV and ADGV are demonstrated to have superior tracking capability of liver fat over time after lifestyle intervention, compared to ALT and AST.

Without being limited by mechanistic theory, the asymmetric form of DMGV, referred to herein as ADGV, is the product of the enzyme alanine glyoxylate aminotransferase 2 (AGXT2), which is believed to catalyse the conversion of asymmetric dimethylarginine (ADMA) to ADGV. However, ADMA is also thought to be converted to citrulline by dimethylarginine dimethylaminohydrolase-1 (DDAH1). Both AGXT2 and DDAH1 enzymes are active in the liver, and their activity is subject to differential regulation. Therefore, ADGV is thought to not be a faithful reporter of AGXT2 activity, as its substrate is also subject to metabolism by another enzyme (DDAH1). In contrast, it is believed that the symmetric form of DMGV, referred to herein as SDGV, is the product of AGXT2 conversion of substrate SDMA, and SDMA is only metabolised by AGXT2. Furthermore, it is thought that SDGV is a faithful reporter of AGXT2 activity, which is believed to be the critical enzyme mediating its role in non-alcoholic fatty liver disease (NAFLD). The present inventors have found that AGXT2 protein and AGXT2 activity are significantly upregulated in NAFLD liver. SDMA, but not ADMA is significantly depleted, and SDGV but not ADGV is significantly upregulated. This demonstrates that the single enzymatic regulation of SDMA, by AGXT2 that converts it into SDGV, confers linear concentration response of SDGV and makes it a faithful reporter of AGXT2 activity, the novel regulator of NAFLD pathology.

### Markers for Liver Fat

Described herein are improved markers for liver fat, specifically the individual symmetric and asymmetric isomers of the metabolite dimethylguanidino valeric acid (DMGV).

DMGV (see, for example, human metabolome database (HMDB) ID: HMDB0240212 https://hmdb.ca/metabolites/HMDB0240212) exists in asymmetric (ADGV) and symmetric forms (SDGV). These isomers differ structurally in that the dimethylated forms of the guanidine functional group are in either in a symmetric or asymmetric orientation. As shown below, the methyl groups in ADGV and SDGV are in different positions.

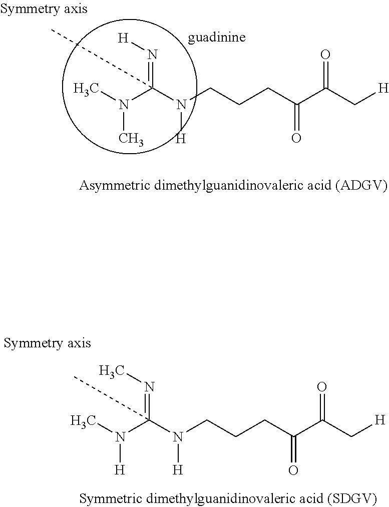

In some embodiments, the markers of the present invention comprise or consist of AGDV. In these embodiments, the AGDV marker can preferably be detected without detecting SDGV in parallel, such that the presence or absence of liver fat in a subject at a given point in time or over series of timepoints is determined based on AGDV level/s rather than level/s of AGDV in combination with SDGV.

In other embodiments, the markers comprise or consist of SDGV. In these embodiments, the SDGV marker can preferably be detected without detecting ADGV in parallel, such that the presence or absence of liver fat in a subject at a given point in time or over series of timepoints is determined based on AGDV level/s rather than level/s of SDGV in combination with AGDV.

In still other embodiments, the markers comprise or consist of AGDV in combination with one or more additional markers. Preferably, the additional marker/s do not comprise SDGV. Non-limiting examples of suitable marker combinations comprising AGDV include: AGDV and ALT, AGDV and AST, AGDV and alkaline phosphatase (ALP), AGDV and albumin, AGDV and albumin and total protein, AGDV and bilirubin, AGDV and gamma-glutamyltransferase (GGT), AGDV and L-lactate dehydrogenase (LD), and AGDV and prothrombin time (PT).

In further embodiments, the markers comprise or consist of SDGV in combination with one or more additional markers. Preferably, the additional marker/s do not comprise AGDV. Non-limiting examples of suitable marker combinations comprising SDGV include: SDGV and ALT, SDGV and AST, SDGV and alkaline phosphatase (ALP), SDGV and albumin, SDGV and albumin and total protein, SDGV and bilirubin, SDGV and gamma-glutamyltransferase (GGT), SDGV and L-lactate dehydrogenase (LD), and SDGV and prothrombin time (PT).

The markers of the present invention can be detected by any suitable means known in the art. Non-limiting examples of suitable techniques include mass spectrometry (MS) (e.g. Tandem Liquid Chromatography Mass Spectrometry/LC-MS/MS), gas chromatography-mass spectrometry (GC-MS/MS), and nuclear magnetic resonance spectroscopy (NMR spectroscopy).

Typically, the markers are detected in a sample, and more preferably a biological sample. For example, the markers may be present in body fluid samples such as blood and its components (e.g. serum, plasma and the like), and/or present in urine. Alternatively, the markers may be present in a tissue sample, such as a liver biopsy. The skilled addressee is well aware of how to obtain various body fluid and tissue samples for testing in accordance with the methods of the invention. The biological sample may be derived from an animal, more preferably selected from a mammal, a non-human mammal, a primate, and a human. Non-limiting examples of suitable subjects from which biological samples for use in the invention can be obtained also include bovine, equine, ovine, avian and rodent species. Alternatively, the sample may be derived from a cell or tissue culture.

### Detection of Liver Fat

The markers of the present invention can be used to detect the presence or absence of liver fat in a subject of interest.

Liver fat accumulation in the subject may arise at least in part from any number of causative factors, non-limiting examples of which include obesity, insulin resistance, metabolic syndrome, excessive alcohol consumption, consumption of certain medications (e.g. steroids, hormones, calcium channel blockers), oxidative stress, dysregulation of gut microbiome, certain infections (e.g. hepatitis C), type 2 diabetes and prediabetes, and so forth.

As demonstrated herein SDGV and ADGV provide superior diagnostic performance in multiple NAFLD/MAFLD patient cohorts. It is also well recognised in the field that fatty liver is a common response to toxicity, regardless of injurious insult. The skilled addressee will thus recognise the general applicability of SDGV and ADGV as individual markers for detecting fat accumulation in any liver injury leading to this complication.

Without particular limitation, detecting the presence or absence of liver fat in a subject using the methods of the invention can provide a means of diagnosing diseases and conditions in subjects characterised by fat accumulation in the liver. Non-limiting examples include fatty liver disease (steatosis), alcoholic fatty liver disease (ALD), non-alcoholic fatty liver disease (NAFLD)/metabolic associated fatty liver disease (MAFLD), and non-alcoholic steatohepatitis (NASH).

The diagnostic methods of the invention are based on detecting liver fat in a subject, by determining the level of a marker or combination of markers as described herein in a sample from the subject. The sample may be a body fluid sample (e.g. blood, serum, plasma) or a tissue sample (e.g. liver tissue).

For example, threshold values of either SDGV or ADGV can be established which are indicative of the presence of absence of liver fat in a subject, based on a normal-abnormal liver fat definition such as percentage weight of fat in total liver weight (e.g. a percentage weight of fat in total liver weight of: more than 15%, more than 10%, more than 5%, between 5% and 15%, between 10% and 15%, between 5% and 10%). By way of non-limiting example, the level of SDGV or ADGV can be measured in a series of samples obtained from a population of subjects having an abnormal weight percentage of liver fat (i.e. above the defined percentage) and from a population of control subjects having a normal weight percentage of liver fat (i.e. above the defined percentage), and the measured levels of SDGV or ADGV used to thereby generate a threshold value allowing discrimination between normal and abnormal liver fat consistent with the defined percentage cut-off.

The liver fat levels in the control subjects can be measured, for example, by standard imaging or biopsy techniques, and/or magnetic resonance spectroscopy using ‘H-MRS’.

The skilled addressee will recognise that multiple approaches may be taken in determining threshold values of either SDGV or ADGV, without requiring inventive skill.

In some embodiments, the level of either SDGV or ADGV in a test sample can be measured and compared with a reference range of SDGV or ADGV levels established from control subjects determined to have certain amounts of liver fat and/or specific form/s of fatty liver disease. This in turn can facilitate a determination of the presence or absence of liver fat and/or normal versus abnormal liver fat and/or the diagnosis of a specific fatty acid disease or condition in the subject from which the test sample was obtained.

In other embodiments, the methods of the present invention can be used to track changes in liver fat within a subject over time. By way of non-limiting example, the level of either SDGV or ADGV can be measured in samples taken at multiple timepoints and compared to threshold values or ranges indicative of liver fat levels. This approach may be taken, for example, to arrive at a prognosis for existing or future liver and/or metabolic disease. Non-limiting examples of such diseases include fatty liver disease (steatosis), alcoholic fatty liver disease (ALD), non-alcoholic fatty liver disease (NAFLD)/metabolic associated fatty liver disease (MAFLD), and non-alcoholic steatohepatitis (NASH). The timepoint sampling may, for example, include baseline, followed by monthly, bimonthly or trimonthly intervals.

In some embodiments, the methods of the present invention further comprise one or more treatment stages to assist in curing the disease, and/or alleviating the symptoms of the disease, and/or alleviating one or more underlying causes of the disease.

The treatments may include, without limitation, prescribing lifestyle intervention (e.g. diet, exercise) to induce weight loss to reduce liver fat, via approaches including any one or more of: energy restriction (e.g. 500-1000 kcal/day), macronutrient composition (e.g. low-to-moderate fat and moderate-to-high-carbohydrate intake), exclusion of fructose intake in beverages and/or foods, limiting of alcohol consumption (e.g. below 30 g for man and below 20 g for women), and/or physical activity (e.g. at least 150-200 min/week of moderate intensity in 3-5 sessions).

Additionally or alternatively, the treatments may include administering to the subject any one or more of insulin sensitisers (e.g. Pioglitazone®), vitamin E, a dual PPARα/δ agonist (e.g. elafibranor), and/or a Farnesoid X receptor (FXR) agonist (e.g. obeticholic acid (OCA)—a 6a-ethyl derivative the human bile acid chenodeoxylcholic acid).

### Detection of Liver Cancer

The markers of the present invention can be used to detect the presence or absence of liver cancer in a subject of interest.

As described herein, taurodeoxycholic acid (TDCA) can be measured and used to identify whether a given subject has liver cancer (i.e. hepatocellular carcinoma), is at increased risk of developing liver cancer, or alternatively determine the absence of liver cancer in a subject of interest.

In some embodiments, the level of TDCA is measured following a determination that the subject has non-alcoholic fatty liver disease (NAFLD). This may improve diagnostic and/or prognostic outcomes in determining the presence or absence of liver cancer in a subject. For example, and without limitation, the subject may be classified as having NAFLD based on the level of SDGV in a biological test sample, such as for example a blood or plasma sample. The subject may then be further tested for TDCA levels in the same biological sample, or a further biological sample.

Accordingly, the methods of the present invention may be used to detect liver fat and/or the presence of liver fat disease such as NAFLD, and/or detect liver cancer/hepatocellular carcinoma.

### Kits

The present invention provides kits comprising one or more instruments and/or reagents for performing methods of the present invention.

In some embodiments the kits may comprise instruments and/or reagents for obtaining samples, for example, body fluid samples such as blood and/or urine, and/or tissue samples such as liver tissue samples.

Additionally or alternatively, the kits may comprise instruments and/or reagents for the processing of samples such as, for example, fractionation of blood into constituent components such as plasma, and/or for the removal of cellular components from blood or urine samples.

Typically, the kits of the present invention may also comprise other reagents, such as wash reagents, enzymes and/or other reagents as required in the performance of the methods of the invention, such as during high performance liquid chromatography and/or mass spectrometry.

The kits may be fragmented kits or combined kits as defined herein.

Fragmented kits comprise reagents that are housed in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion.

Such kits may also include a container which will accept the test sample, a container which contains the reagents used in the assay, containers which contain wash reagents, and containers which contain a detection reagent.

Combined kits comprise all of the components of a reaction assay in a single container (e.g. in a single box housing each of the desired components).

The kits of the present invention may also include instructions for using kit components to conduct the appropriate methods. Kits and methods of the invention may be used in conjunction with automated analysis equipment and systems, for example, including but not limited to, high performance liquid chromatography systems and/or mass spectrometers.

### Exemplary Detection Method for SDGV or AGDV

By way of non-limiting example only, SDGV or AGDV can be detected via high performance liquid chromatography-mass spectrometry (HPLC-MS) in accordance with certain embodiments of the invention.

An ultra high-performance liquid chromatography (UHPLC) system may be used such as, for example, a Shimadzu Nexera LC-30AD comprising a binary pump, an isocratic pump, an autosampler, and a thermostatted column compartment equipped with a six-port switching valve. The chromatographic separation may take place on a suitable column such as, for example, a Hypercarb 50×2.1-mm column with a particle size of 5 μm protected by a 10×2.1-mm precolumn filled with the same material (ThermoFisher Scientific, San Jose, CA, USA). Online cleanup of the samples may be performed using a suitable column, for example a Zorbax Eclipse XDB-CN 50×2.0-mm column with a particle size of 5 μm (Agilent). Mass spectrometric detection can be performed using any appropriate mass spectrometer (e.g. on a Sciex 6500+ triple quadrupole mass spectrometer (AB Sciex, Framingham, MA, USA)).

In specific embodiments, and again by way of non-limiting example, the HPLC system may be configured with two pumps and a six-port switching valve. In valve position 1, the sample can be directed to the cleanup column and subsequently to the analytical column. After, for example, 1.5 min, the valve can be switched to position 2. In this position, the binary pump via the autosampler can be connected directly to the analytical column, whereas the isocratic pump can be configured to back-flush the cleanup column. Mobile phase A can be aqueous buffer (e.g. 0.1% formic acid and 0.1% ammoniumformate, resulting in pH ˜3.5), whereas mobile phases B and C may be acetonitrile. Mobile phase A and B can be connected to the binary pump, and mobile phase C may be connected to the isocratic pump. A gradient elution using the binary pump can be applied starting at 100% mobile phase A. After, for example, 1 minute, the ratio of mobile phase B can be raised to around 20% in about 10 minutes and held constant for about 4 minutes. After a total run time of about 15 minutes, the valve can be switched back to position 1 and the columns reequilibriated with 100% mobile phase A for about 6 minutes. The columns can be held at a constant temperature of about 40° C., using an injection volume of about 25 μl. Mass spectrometric detection can be carried out applying electrospray ionization (ESI) and selected reaction monitoring. The ion source may be working in the positive mode at an ionization voltage of, for example, 4.5 kV. Sheath gas and auxiliary gas (both nitrogen) can be set to 30 and 10 arbitrary units, respectively. The temperature of the transfer capillary may be at around 260° C. Under these conditions, the [M+H]⁺ quasimolecular ions m/z 202.1 and 208.1 of ADGV/SDGV and D₆-ADGV/SDGV, respectively, can be formed, substantially or entirely without formation of dimers, adduct ions, or source fragmentation products. The quasimolecular ions can be fragmented in the collision cell of the mass spectrometer at, for example, 22 eV with argon at a pressure of 1.3 mTorr serving as collision gas. From the resulting fragment ions, m/z 70.1 and 76.1 for ADGV/SDGV and D₆-ADGV/D₆-SDGV, respectively, can be observed for quantification, and m/z 46.1 recorded for qualification for both substances. ADGV may have a retention time of 11.75 minutes and SDGV a retention time of about 12.5 minutes.

By way of non-limiting example only, the following approach may be taken for the preparation of calibration standards and QC samples. For plasma calibration, aqueous calibration samples (e.g. ×6) in the concentration range from about 10 to 200 nmol/L can be prepared. In an analogous way, for urine calibration, aqueous calibration samples (e.g. ×6) in a concentration range from about 0.1 to 20 μmol/L can be prepared. Quality control (QC) samples can be prepared in three concentrations from both human plasma and urine. The QC-low level for plasma may be blank human plasma without any spiking. The QC-medium and QC-high levels can be prepared by spiking the same plasma with about 50 and 200 nmol/L ADGV/SDGV, respectively. All QC samples can be stored (e.g. at −80° C.) until use.

Samples may be prepared according to the following non-limiting/exemplary method: 20 μL of plasma, 80 μL of HILIC IS-IS [consists of acetonitrile:methanol:formic acid (75:25:0.2, v:v:v) and 250 nmol/L D₆-ADGV or D₆-SDGV, cooled to −30° C.] can be combined to a final volume of 100 μL. Samples may be vortexed to promote protein precipitation and centrifuged at about 14000 rpm for about 20 min at about 4° C. A total of about 75 μL of supernatant can be transferred into glass vial with inserts, taking care to avoid transferring protein pellet particles. Each vial may be capped tightly and store at about −30° C. (or about 10° C. in the autosampler stack).

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

Exemplary Diagnostic and/or Prognostic Methods for Liver Cancer

By way of example only and without limitation, an exemplary method according to the present invention may be carried out as follows.

A test subject of interest may be tested for the presence of liver fat using the methods described herein. A biological sample from the subject such as, for example, blood or plasma extracted from blood, may first be measured for SGDV concentration.

In some embodiments, the concentration of SDGV measured in the plasma sample may be below 0.3 μM, below 0.4 μM, below 0.5 μM, below 0.51 μM, below 0.515 μM, below 0.516 μM, or below 0.52 μM. In these embodiments, the subject may be determined to have acceptable levels of liver fat, and it may be determined on that basis that NAFLD and HCC are not present.

In other embodiments, the concentration of SDGV measured in the plasma sample may be above 0.8 μM, above 0.7 μM, above 0.6 μM, above 0.55 μM, above 0.54 μM, above 0.53 μM, above 0.52 μM, or above 0.516 μM. In these embodiments, the subject may be determined to have elevated levels of liver fat, and it may be determined on that basis that NAFLD and potentially HCC are present.

In embodiments where it is determined that NAFLD and potentially HCC are present, the subject may be further tested for TDCA levels. The same or a different biological sample may be tested for TDCA concentration, for example blood or plasma extracted from blood.

In some embodiments the TDCA concentration measured in the plasma may be below 0.75 μM, below 0.7 μM, below 0.65 μM, below 0.6 μM, or below 0.5 μM. In these embodiments, the subject may be determined to be a risk of developing HCC.

In other embodiments the TDCA concentration measured in the plasma may be above 0.65 μM, above 0.7 μM, above 0.75 μM, above 0.8 μM, or above 0.85 μM. In these embodiments, the subject may be determined to be have HCC.

The skilled person will recognise that the above embodiments are exemplary only and may be adapted to suit specific clinical settings as needed using standard knowledge in the field.

### Examples

The present invention will now be described with reference to specific Example, which should not be construed as in any way limiting.

### Example One: Symmetric Dimethylguanidino Valeric Acid (SDGV) is a Superior Diagnostic Marker for Liver Fat

### Introduction and Aims

The accurate detection of fatty liver disease remains a diagnostic challenge. The metabolite dimethylguanidino valeric acid (DMGV) has been identified as a circulating biomarker of metabolic associated fatty liver disease (MAFLD).

As identified in this study DMGV consists of two isomers-symmetric (SDGV) and asymmetric (ADGV). This study sought to:
1) determine the diagnostic performance of asymmetric (ADGV) and symmetric dimethylguanidino valeric acid (SDGV)—compared to fatty liver index (FLI), alanine aminotransferase (ALT), and aspartate aminotransferase (AST)—in plasma samples from participants of the prediabetes lifestyle intervention study (PLIS) who were randomized to dietary intervention: no intervention; conventional intervention; or intensive intervention, with liver fat quantified by MRI;
2) assess ADGV's relationship to liver fat histological characteristics in a Sydney hospital-based cohort with patients who span the spectrum from mild “simple” steatosis to advanced MAFLD with fibrosis;
3) investigate the involvement of AGXT2 and its metabolites in a mouse model of MAFLD.

MAFLD diagnostic criteria have been shown to be more accurate than NAFLD for predicting disease progression in the National Health and Nutrition Examination Survey (NHANES) cohort.^{12,13} Therefore, in this Example MAFLD terminology is used, the accepted histopathology definitions such as steatosis, hepatocyte ballooning, fibrosis are included, and the comorbid metabolic conditions such as hypertension, diabetes mellitus, and dyslipidaemia are indicated. For clarity, the NAFLD activity score (NAS) is also presented whilst terminology is in evolution.

### Methods

In two cohorts—one in Sydney, Australia, and the prediabetes lifestyle intervention study (PLIS) cohort in Dresden, Germany—plasma ADGV, SDGV, related metabolites, and standard plasma markers of MAFLD were measured including aspartate aminotransferase (AST) and alanine aminotransferase (ALT), and compared their diagnostic performance for liver fat detection.

### Description of the Cohorts

### PLIS Cohort-Dresden

The prediabetes lifestyle intervention study (PLIS) is a stratified-randomized, controlled multi-center trial involving eight study sites in Germany (ClinicalTrials.gov Identifier: NCT01947595). The primary hypothesis of the study is that individuals with prediabetes who are high-risk for failure to restore normal glucose regulation using conventional lifestyle intervention (LI) will benefit from an intensification of the LI. Prediabetes was diagnosed from fasting and 2-hour post-challenge glucose levels after a standardized oral glucose tolerance test (OGTT), according to the criteria of the American Diabetes Association. Screening procedures also involved measurement of liver fat content, insulin sensitivity, and insulin secretion. For the current study, 213 participants from the Dresden site who had liver fat determined at two timepoints 1 year apart were included. A full description is provided in¹⁴.

### Sydney MAFLD Cohort

This cohort consists of patients referred to a tertiary hepatology service on the basis of imaging evidence of steatosis (usually ultrasound), and/or abnormal plasma liver function tests. Plasma samples from a total of 193 patients with biopsy-proven MAFLD were used in the analysis. The cohort was on average overweight with an average BMI of 29.12; 24.4% had hypertension, 17.6% had diabetes mellitus, and 37.8% had dyslipidaemia. More than half the cohort had moderate to severe fibrosis (F2-4, 59.6%). Moderate to severe steatosis was present in the majority of the cohort (127 patients (65.8%)), however, the majority of the cohort had low NAFLD activity scores (NAS) of less than 5 (165 patients, 85.5%). Fibrosis, steatosis, portal inflammation, NAS were assessed using a scoring system, where components are graded on a scale for steatosis (0-3), lobular inflammation (0-2), hepatocellular ballooning (0-2), and fibrosis (0-4). The NAS score is the sum of scores for steatosis, inflammation and ballooning, and does not include the stage for fibrosis.

### Statistical Analysis

Prior to statistical modelling, both the biomarkers and the liver fat measurements were pre-processed by applying a logarithm to remedy their right-skewness. The biomarkers on log-scale were then mean-centred and scaled to have unit variance for better comparability between the biomarkers. Linear mixed models were used to quantify the relationship between a biomarker and liver fat as dependent variable. As clinically relevant covariates besides the biomarker itself, age, sex, waist-to-hip ratio (WHR), body mass index (BMI), and intervention arm were incorporated. The predictor variables WHIR, BMI and the biomarker were measured twice. and therefore relationships with baseline and change overtime were assessed, A random intercept effect on patient level accommodates the correlation of the repeated liver fat measurements per patient. The model assumptions were checked by means of diagnostic plots (residual plots, QQ-plots) for each regression model. Statistical significance of a predictor in the model was determined by likelihood ratio tests. For cut-off, sensitivity, specificity, positive predictive value, and negative predictive value, the cut-off level of MR defined-FEW was used as originally described by¹⁰.

### Metabolite Measurements in PLIS Cohort and Mice

Levels of AGXT2 substrates ADMA, SDMA, beta-aminoisobutyric acid (BAIBA), L-homoarginine and L-arginine and products ADGV, SDGV and 6-guanidino-2-oxocaproic acid (GOCA) were measured in plasma of the PLIS cohort patients and in mouse plasma by isotope-dilution tandem mass spectrometry (LC-MS/MS) as previously described.^{15,16}

### Metabolite Measurement in Sydney MAFLD Cohort

Levels of ADGV, BAIBA, Arginine, and Citrulline were measured using liquid chromatography-tandem mass spectrometry (LC-MS/MS) on an Agilent 1260 Infinity HPLC System (Agilent Technologies, Santa Clara, CA, USA) coupled to an AB SCIEX QTRAPVR 5500 μMS tandem mass spectrometer (MS/MS) triple quadrupole (QqQ) mass analyser operating in MRM scan mode in positive ion mode using an AtlantisVR HILIC column. Samples were randomized and an internal pooled sample was run every 10 samples for quality control. All raw data files (Analyst software, version 1.6.2; AB Sciex, Foster City, CA, USA) were imported into Muiti-Quant™ 3.0 Software for MRM Q1/Q3 peak integration. To account for any performance drift in the LC-MS/MS, the metabolite abundance in each sample was normalized to the bookended pool plasma sample (every 10 samples), deriving a ‘Normalized area (AU)’ (normalized abundance) for each metabolite per standard practice.

### Murine Model

To determine further the relationship of liver fat accumulation to expression of the generative pathway for these biomarkers, a murine model was used. The db/db leptin receptor-deficient mouse model is one of the most common and well-established murine models of diabetes mellitus.¹⁰ Along with obesity this model demonstrates all the key biochemical and physiological features of metabolic abnormalities associated with MAFLD: fasting hyperglycaemia, hyperinsulinaemia and dyslipidaemia and shows micro- and macrovesicular steatosis, which makes it particularly useful for the study of mechanisms and possible treatment options for MAFLD.¹¹

All animal experiments were approved by local authorities (approval No.: DD24.1-5131/476/3). Six month old db/db (n=10) and db/+ (n=15) male mice (the Jackson Laboratory BKS.Cg-Dock7^{m}+/+Lepr^{db}/J; stock no. 000642) were used for the experiments. All animals were housed at constant humidity (60±5%), temperature (24±1° C.), and a 12 h light/dark cycle (6 AM to 6 PM light). Mice had unlimited access to water and food.

### Collection of Plasma and Organs

Mice were subjected to isofluorane anaesthesia and blood was collected by cardiac puncture into EDTA containing tubes (final concentration 5 mmol/L). Mice were subsequently perfused with 0.9% (w/v) NaCl solution. Liver and kidneys were harvested, flash-frozen and stored at −80° C. until further analysis. Plasma was separated by centrifugation and stored at −80° C.

### Histochemistry

Immediately after isolation liver samples were fixed in cold 4% paraformaldehyde diluted in phosphate-buffered saline at 4° C. and processed for paraffin embedding, cross-sectioned to obtain 4 μm-thick sections and mounted on glass slides. Tissue sections were deparaffinized in xylene 3×5 min and rehydrated in descending concentrations of ethanol (100%, 96%, 70% and 40%, 2 min each). The slides were placed in haematoxylin solution for 10 min, rinsed with running tap water for 10 min and immersed in distilled water for 1 min. Next, the slides were put in eosin solution for 2 min, rinsed three times in distilled water, dehydrated in ascending concentrations of ethanol (40%, 70%, 96% and 100%, 2 min each), cleared in xylene 3×2 min and mounted with DePeX medium. The photos were taken with Zeiss Apotome microscope (Germany) and analysed in ImageJ, version 1.53 c (National Institute of Health).

### Detection of AGXT2 Protein Levels and Activity

Detection of AGXT2 protein in kidney and liver lysates from db/db mice was performed by immunoblotting using rabbit anti-AGXT2 antibody (Sigma-Aldrich #HPA037382, dilution 1:2000), which recognizes both mouse and human AGXT2 using a previously established protocol.¹⁷ μMeasurement of AGXT2 activity in tissues from db/db mice was performed using stable isotope-labelled ADMA as substrate with subsequent detection of labelled ADGV as previously described.¹⁸

### Results

Both ADGV and SDGV were measured in the PLIS cohort, and were diagnostically superior to AST and ALT, including ability to track liver fat changes over time, with SDGV performing the best. ADGV and SDGV were diagnostically equal to the fatty liver index.

In a murine model of MAFLD, AGXT2 protein level and specific activity were significantly increased; SDGV and its substrate were significantly changed whereas ADGV and its substrate were not, affirming SDGV as a faithful reporter of AGXT2 activity in MAFLD, underpinning its superior diagnostic performance.

Hence, these results demonstrate an improved diagnostic performance of a new blood biomarker of fatty liver disease compared to the current standard of care. The new biomarker, SDGV, performed better in two domains necessary for a good biomarker:
1) improved cross-sectional performance;
2) improved tracking capacity over time.

In particular, SDGV can be used widely to improve detection of liver fat and to track efficacy of new treatments for MAFLD.

### Baseline Characteristic of the Cohorts

The baseline characteristics of the participants of the PLIS study and the Sydney-based MAFLD cohort are listed in Table 1 and Table 2, respectively.

TABLE 1
PLIS cohort characteristics.

| Variable | Value |
|---|---|
| Women/men, n (%) | 81/132 (38/62%) |
| Age [years, mean ± SD] | 65 ± 8 |
| Body Mass Index [kg/m², mean ± SD] | 28.43 ± 4.24 |
| Waist-to-hip ratio [cm/cm, mean ± SD] | 0.92 ± 0.08 |
| Visceral fat mass_{MRI} [l, mean ± SD] | 5.73 ± 2.41 |
| Intrahepatic lipid content_{MRI} [%-abs, mean ± SD] | 15.21 ± 8.51 |
| Fasting glucose [mmol/L, mean ± SD] | 6.1 ± 0.4 |
| ALT [IU/L, mean ± SD] | 35.94 ± 26.64 |
| AST [IU/L, mean ± SD] | 29.91 ± 19.03 |
| HbA1c [mmol/mol] | 39.94 ± 3.16 |
| 2-h glucose [mmol/L, mean ± SD] | 7.8 ± 1.8 |
| Fasting insulin [pmol/L, mean ± SD] | 107.1 ± 63.1 |
| 2-h insuline [pmol/L, mean ± SD] | 575.9 ± 467.1 |
| Triglycerides [mmol/L, mean ± SD] | 1.4 ± 0.9 |
| Total cholesterol [mmol/L, mean ± SD] | 5.42 ± 1.05 |
| HDL-cholesterol [mmol/L, mean ± SD] | 1.52 ± 0.61 |
| LDL-cholesterol [mmol/L, mean ± SD] | 3.07 ± 0.82 |
| HOMA-IR [mean ± SD] | 4.71 ± 2.97 |

SD: standard deviation; IU/L: international units/Litre; mmol/L: millimoles/Litre; pmol/L: picomoles/Litre; MRI: magnetic resonance imaging; ALT: alanine aminotransferase; AST: aspartate aminotransferase; HbA1c: glycated haemoglobin; HDL: high-density lipoprotein; LDL: low-density lipoprotein; HOMA-IR: homeostatic model assessment of insulin resistance.

TABLE 2
Sydney-based MAFLD cohort characteristics.

| Variable | Value |
|---|---|
| Age, yrs (mean ± SD) | 50 ± 32 |
| Male n (%) | 107 (61) |
| BMI, kg/m² (mean ± SD) | 29.12 ± 14.73 |
| ALT, IU/L | 72.62 ± 13.44 |
| AST, IU/L | 52.04 ± 15.56 |
| Triglycerides, mmol/L | 1.9 ± 1.05 |
| Total cholesterol, mmol/L | 5.13 ± 2.4 |
| HDL-cholesterol, mmol/L | 1.27 ± 0.43 |
| LDL-cholesterol, mmol/L | 3.06 ± 1.05 |
| HOMA-IR | 4.52 ± 1.07 |
| STEATOSIS | |
| None and mild, n (S0-S1) (%) | 66 (34.2%) |
| Moderate and severe, n (S2-S3) (%) | 127 (65.8%) |
| HEPATOCYTE BALLOONING | |
| None and mild (0-1), n (%) | 101 (52.3%) |
| Severe (2), n (%) | 92 (47.7%) |
| NAS | |
| <5, n (%) | 165 (85.5%) |
| ≥5, n (%) | 28 (14.5%) |
| FIBROSIS SCORE | |
| F0-1 | 78 (40.4%) |
| F2-4 | 115 (59.6%) |
| METABOLIC COMORBIDITIES | |
| Hypertension, n (%) | 47 (24.4%) |
| Diabetes mellitus, n (%) | 34 (17.6%) |
| Dyslipidemia, n (%) | 73 (37.8%) |

SD: standard deviation; BMI: body-mass index; IU/L: international units/Litre; mmol/L: millimoles/Litre; ALT: alanine aminotransferase; aspartate aminotransferase; HDL: high-density lipoprotein; LDL: low-density lipoprotein; HOMA-IR: homeostatic model assessment of insulin resistance; NAS: NAFLD (non-alcoholic fatty liver disease) activity score.

### Reductions in Liver Fat Tracked Reductions in BMI

In the PLIS human cohort comprising 213 individuals with varying degrees of liver fat measured at two timepoints 1 year apart, liver fat was determined using MRI Fat-Free Weight (FFW), the most accurate modality available, with normal levels determined as originally described by¹⁰.

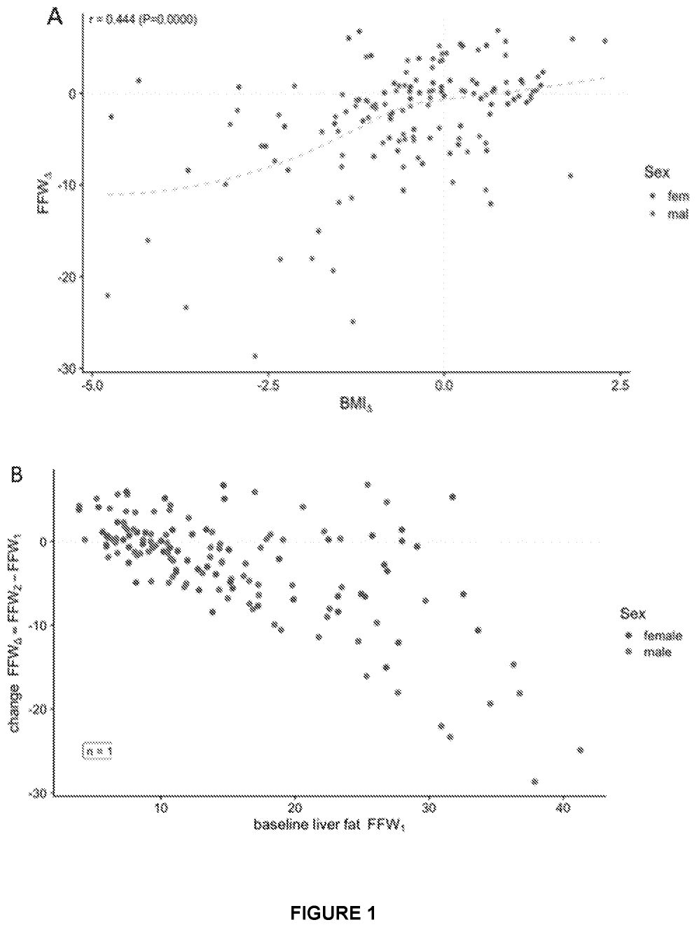

It was first determined whether successful weight loss led to reduction in liver fat content in the PLIS participants. Change in BMI was compared to change in liver fat content. As expected, changes in BMI (BMI_{Δ}) correlated with changes in liver fat (FFW_{Δ}) (FIG. 1A). Following this the correlation between change in liver fat over the follow-up period (FFW_{Δ}) and the amount of liver fat at baseline (FFW₁) was investigated. As can be seen in FIG. 1B, the greater the baseline liver fat content, the greater the reduction in liver fat.

### SDGV and ADGV Detect Liver Fat Cross-Sectionally and Track Liver Fat Change Over Time

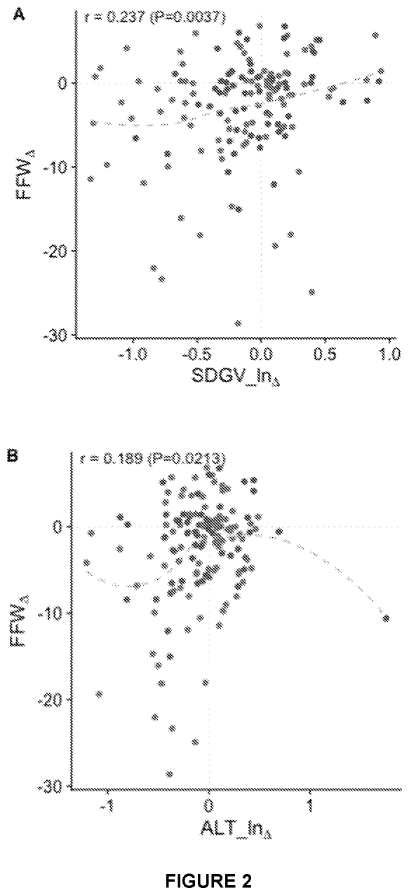

A comparison was made of “change in biomarker vs change in liver fat”, for SDGV and also for ALT, the current best biomarker of NAFLD, illustrated in FIG. 2.

Change in blood SDGV levels correlated better (r=0.24, P=0.0037) with change in MRI FFW than did blood ALT (r=0.19, P=0.02), the current best biomarker of liver fat. However, this crude correlation of delta vs delta has been criticized as a less rigorous test of biomarker performance, due to the major confounder “regression to the mean”: those with the greatest baseline liver fat have the greatest change in liver fat over time. Regression to the mean weighs in favour of the most affected individuals, and confounds assessment of the full spectrum of liver fat, i.e. those with mild, moderate, and severe accumulation of liver fat.

To address this problem, and to more accurately test liver fat “tracking” capability of each biomarker, a linear mixed model was employed that keeps the absolute level of biomarker and of liver fat independent from change in biomarker and liver fat. The results are plotted in Table 3, “Biomarker Change”. As clinically relevant covariate age, sex, body-mass index (baseline level and change during follow-up), waist-to-hip ratio (baseline level and change during follow-up), and type of intervention were incorporated. As the ultimate biomarker would perform well in cross-sectional association at a particular timepoint (Table 2 “Biomarker Average”) AND association with change over time, both were assessed. As can be seen Table 3, only 2 blood biomarkers had a consistent relationship with FFW when assessing both change in average biomarker levels and deviation from the mean biomarker level: SDGV and ADGV, with SDGV performing far better. Furthermore, change in ALT or AST, the currently used blood biomarkers for detection of liver fat, were not significantly associated with change in liver fat, demonstrating their inability to “track” liver fat change over time.

TABLE 3
Effect of average biomarker levels and change in biomarker levels on liver fat

| | Biomarker Average | | | Biomarker Change | | | Model Fit |
|---|---|---|---|---|---|---|---|
| Biomarker | Increase_{avg} | Effect on FFW | P_{avg} | Increase_{chg} | Effect on FFW | P_{chg} | R²_{marg} |
| SDGV | +80.40% | +19.88% | <0.001 | +12.75% | +02.66% | <0.001 | 0.1566 |
| ADGV | +85.89% | +17.94% | <0.001 | +13.88% | +02.40% | 0.0034 | 0.1358 |
| ALT | +53.73% | +24.59% | <0.001 | +09.42% | +01.57% | 0.0784 | 0.2139 |
| GOCA | +84.04% | +13.69% | <0.001 | +19.72% | +01.19% | 0.1412 | 0.0884 |
| AST | +32.31% | +13.09% | <0.001 | +07.25% | +00.84% | 0.3802 | 0.0829 |
| HomoArg | +40.49% | +07.40% | 0.051 | +05.13% | +00.49% | 0.5568 | 0.0437 |
| Arginin | +18.53% | −04.26% | 0.241 | +03.05% | −01.17% | 0.1537 | 0.0334 |
| ADMA | +12.75% | +02.85% | 0.436 | +03.05% | +01.12% | 0.1603 | 0.0293 |
| SDMA | +20.92% | −02.22% | 0.599 | +03.05% | +00.12% | 0.8896 | 0.0257 |

SDGV: symmetric α-keto-dimethylguanidinovaleric acid; ADGV: asymmetric α-keto-dimethylguanidinovaleric acid; BAIBA: beta-aminoisobutyric acid; ADMA: asymmetric dimethylarginine; GOCA: 6-guanidino-2-oxocaproic acid; hARG: homoarginine; SDMA: symmetric dimethylarginine; P = pvalue; R² = correlation coefficient.

SDGV has Better “Rule in/Rule Out” Capacity for Liver Fat than Standard Blood Biomarkers

Analyses were performed to test the ability of each biomarker to detect and to rule-out liver fat at a single timepoint (Table 4). As SDGV performed best, its ability to determine NAFLD was compared to current standard of care, ALT and AST. As seen in Table 4, SDGV performed better than both currently-used blood markers of liver fat. SDGV was able to:
a) detect liver fat with greater sensitivity than ALT and AST
b) rule out liver fat with a stronger negative predictive value than ALT and AST.

TABLE 4
Diagnostic performance of SDGV, ALT, and AST

| Biomarker | Cut-off | Cut-off percentile | Sensitivity | NPV |
|---|---|---|---|---|
| SDGV | 4.97 | 0.377 | 0.7 | 0.33 |
| ALT | 3.3 | 0.45 | 0.6 | 0.31 |
| AST | 3.2 | 0.47 | 0.5 | 0.2 |

SDGV: symmetric dimethylguanidino valeric acid; ALT: alanine aminotransferase; AST: aspartate aminotransferase; NPV: negative predictive value.

### ADGV and BAIBA are Predictors of Fatty Liver Disease and Fibrosis in the Sydney MAFLD Cohort

ADGV, but not SDGV, was measured in the Sydney MAFLD cohort, along with related metabolites beta-aminoisobutyric acid (BAIBA), arginine, and citrulline. ADGV was significantly associated with fibrosis (Table 5) and steatosis (Table 6). BAIBA was inversely associated with portal inflammation (Table 6) and NAFLD activity score (NAS) (Table 7). Arginine and Citrulline were not significantly associated with any parameters.

TABLE 5
Correlation of AGTX2 metabolites with
fibrosis in Sydney MAFLD cohort.

| Metabolite | Correlation Coefficient | Adjusted P-value |
|---|---|---|
| ADGV | 0.22 | 2.6 × 10⁻³ |
| BAIBA | 0.06 | 0.40 |
| Arginine | −0.03 | 0.66 |
| Citrulline | −0.02 | 0.83 |

ADGV: asymmetric α-keto-dimethylguanidinovaleric acid; BAIBA: beta-aminoisobutyric acid. Fibrosis was assessed using a scoring system.

TABLE 6
Correlation of AGTX2 metabolites with
steatosis in Sydney MAFLD cohort.

| Metabolite | Correlation Coefficient | Adjusted P-value |
|---|---|---|
| ADGV | 0.16 | 3.6 × 10⁻² |
| BAIBA | −0.09 | 0.22 |
| Arginine | −0.01 | 0.42 |
| Citrulline | −0.03 | 0.74 |

ADGV: asymmetric α-keto-dimethylguanidinovaleric acid; BAIBA: beta-aminoisobutyric acid. Steatosis was assessed using a scoring system.

TABLE 6
Correlation of AGTX2 metabolites with portal
inflammation in Sydney MAFLD cohort.

| Metabolite | Correlation Coefficient | Adjusted P-value |
|---|---|---|
| BAIBA | −0.27 | 6.5 × 10⁻⁴ |
| ADGV | 0.10 | 0.23 |
| Arginine | −0.09 | 0.26 |
| Citrulline | −0.04 | 0.67 |

ADGV: asymmetric α-keto-dimethylguanidinovaleric acid; BAIBA: beta-aminoisobutyric acid. Portal inflammation was assessed using a scoring system.

TABLE 7
Correlation of AGTX2 metabolites with NAFLD-
activity score in Sydney MAFLD cohort.

| Metabolite | Correlation Coefficient | Adjusted P-value |
|---|---|---|
| BAIBA | −0.18 | 1.6 × 10⁻² |
| ADGV | 0.12 | 0.11 |
| Arginine | −0.08 | 0.26 |
| Citrulline | −0.06 | 0.40 |

ADGV: asymmetric α-keto-dimethylguanidinovaleric acid; BAIBA: beta-aminoisobutyric acid. NAFLD-activity score was assessed using a scoring system.

### SDGV and ADGV are Equal to Fatty Liver Index (FLI) in Predicting Liver Fat in the PLIS Cohort

It was next investigated how the diagnostic performance of the biomarkers compared with fatty liver index (FLI: comprising body mass index, waist circumference, plasma triglycerides and gamma-glutamyl-transferase) in predicting liver fat. First, no significant difference was found between the correlations of: SDGV with liver fat and FLI with liver fat (p=0.38); or between the correlations of ADGV with liver fat and FLI with liver fat (p=0.18). For the other biomarkers, the correlations with liver fat were significantly lower than that of FLI.

Second, the regression coefficients of SDGV and ADGV, when adding FLI as a covariate in the linear model, decreased by 0.075 (SDGV) and 0.053 (ADGV), suggesting that SDGV and ADGV's diagnostic performance is orthogonal to that of FLI.

Third, the diagnostic performance of FLI was improved in the multivariate model when SDGV or ADGV were added, as assessed using the residual standard error (sigma) and the Akaike information criterion (AIC). SDGV decreased sigma from 0.508 to 0.476, and AIC from 245.9 to 225.2; ADGV decreased sigma from 0.518 to 0.475, and AIC from 252.0 to 224.7.

The above assessments imply:
1) non-inferiority for SDGV and ADGV compared to FLI for diagnosis of liver fat;
2) additive diagnostic performance when SDGV or ADGV are combined with FLI.
   ADGV is a Better Predictor of Liver Fat than FLI in the Sydney Cohort

A similar analysis in the Sydney cohort revealed that ADGV (standardized beta coefficient=0.17, p=0.038) could significantly predict the presence of liver fat whereas FLI could not (standardized beta coefficient=0.12, p=0.13). In a multivariate model for detection of steatosis, addition of ADGV levels to the FLI improved the standardized beta coefficient to 0.15 and reduced the Akaike information criterion (101.37 before vs 100.14 after addition of ADGV).

### SDGV, not ADGV, is a Faithful Reporter of Liver AGXT2 Activity in a Mouse Model of MAFLD

The analyses above examined correlation and diagnostic performance of ADGV and SDGV. It was next determined changes in the enzyme that produces both these metabolites, called alanine:glyoxylate aminotransferase 2 (AGXT2), which is mostly expressed in the liver and kidney. db/db leptin-deficient mice were used as a model of obesogenic type 2 diabetes and MAFLD.

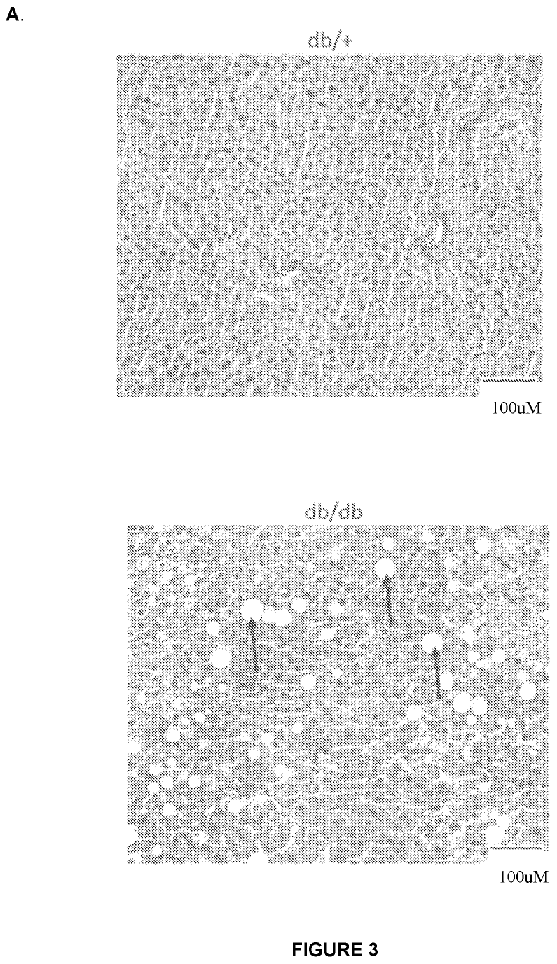

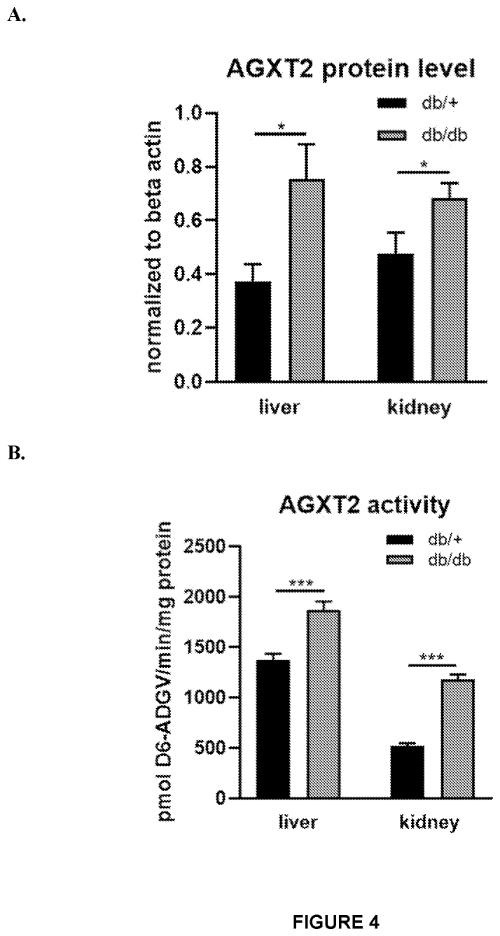

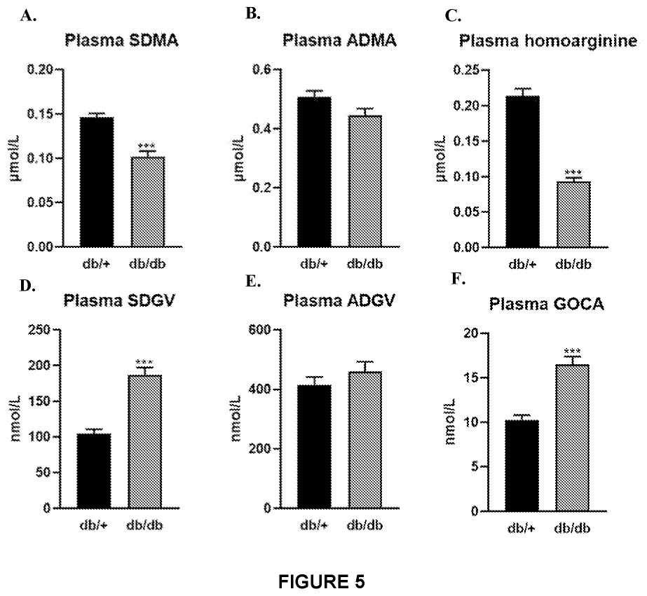

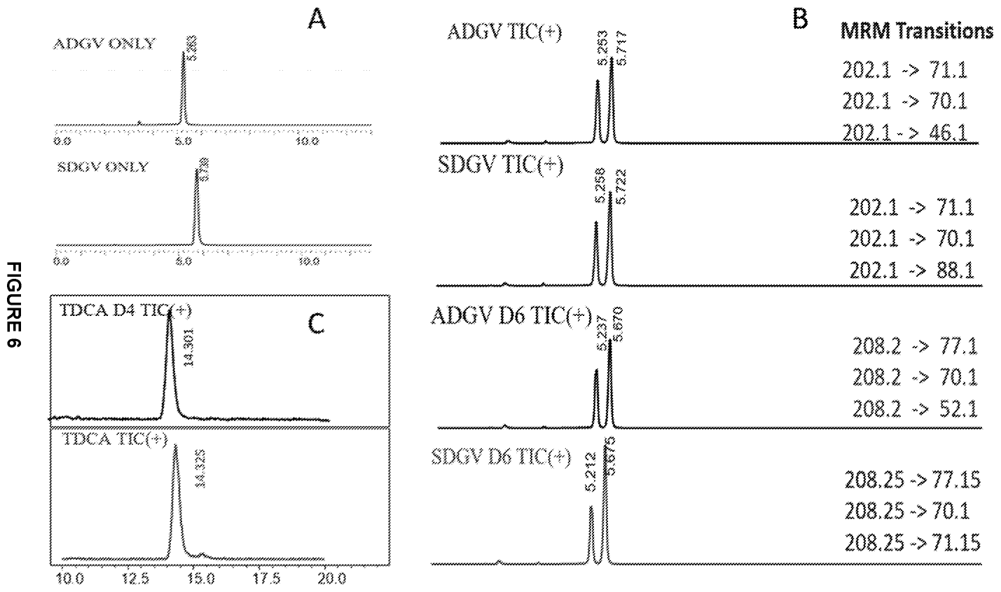

6-month-old mice were sacrificed, liver histology performed, the amount of AGXT2 protein in the kidney and liver quantified, and specific enzyme activity of AGXT2 in those tissues measured. Liver histology confirmed presence of mediovesicular hepatic steatosis in db/db mice. Obese db/db mice demonstrated excessive lipid accumulation in the liver in comparison to control db/+ animals indicating the presence of MAFLD (FIG. 3). AGXT2 protein levels in the liver and kidney were significantly higher in the db/db than in the db/+ mice (FIG. 4A), as was AGXT2 specific activity (FIG. 4B). In line with this data, when the AGXT2 substrates and products in the plasma were measured, it was discovered that the concentrations of ADMA and homoarginine (AGXT2 substrates) were lower in the db/db than in the db/+ mice and the concentrations of GOCA and SDGV (AGXT2 products) were increased in db/db compared to db/+ mice. The differences in the concentrations of ADMA and ADGV did not reach statistical significance, however those of SDMA and SDGV did (FIG. 5).

### Discussion

The main findings of this study are:
1) plasma levels of SDGV and ADGV are superior plasma markers of liver fat than the currently used biomarkers, ALT and AST;
2) SDGV and ADGV are equally, or more, discriminatory for liver fat than FLI; 3) adding SDGV and ADGV to FLI increases its predictive ability; and
4) the superior diagnostic performance of SDGV compared to ADGV likely reflects the fact that SDGV is a faithful reporter of AGXT2 activity in the liver.

These results investigated the prognostic utility of the component metabolites of DMGV-ADGV and SDGV, and demonstrate equal or superior discriminatory capacity as FLI. FLI is a well-known, widely accepted surrogate index of liver fat which was introduced in 2006 and is based on body mass index, waist circumference, plasma triglycerides, and gamma-glutamyl-transferase levels.¹⁹ An FLI <30 may be used to rule out NAFLD and FLI ≥60 suggests presence of the disease. Previous studies validated the usefulness of this index in predicting fatty liver¹⁹ making it a commonly used index in the clinic.²⁰ However, there is also published data available demonstrating that the fatty liver index does not perform well enough to quantify the actual risk of fatty liver disease²¹, does not accurately quantify steatosis, and is confounded by inflammation and fibrosis.²²

In the current study a putative mechanism of elevated levels of ADGV and SDGV in fatty liver disease was provided. Specifically, it was shown that db/db mice, an established model of MAFLD and diabetes, have increased protein and specific activity levels of AGXT2 in the liver and kidney. Using histochemical staining in liver slides the data presented here shows that the db/db mice have increased amount of liver fat compared to the control mice. AGXT2 has multiple enzymatic activities,²³ with ADGV and SDGV being the products of catabolism of asymmetric dimethylarginine (ADMA) and symmetric dimethylarginine (SDMA), respectively. In line with increased protein levels and enzymatic activity, it was demonstrated that levels of SDMA and homoarginine (substrates of AGXT2) were decreased in the plasma of db/db mice and that levels of corresponding products (SDGV and 6-guanidino-2-oxocaproic acid) were increased. A significant difference in the plasma levels of ADMA and ADGV between the db/db and db/+ mice was not observed. A possible explanation could be that the levels of ADMA are regulated by proteolysis and metabolized by both AGXT2 and another enzyme, dimethylarginine dimethylaminohydrolase (DDAH).²⁴ Conversely, SDGV is generated by single enzymatic conversion of SDMA by AGXT2. Therefore, the superior diagnostic performance of SDGV is putatively linked to its faithful reporting of AGXT2 activity.

### Example Two: Normative Upper Limit of Normal (ULN) for SDGV and Ability of TDCA to Distinguish NAFLD-Derived Hepatocellular Carcinoma (HCC) from Non-HCC

### Materials and Methods

### Standards and Reagents

ADGV, SDGV and the isotope-labelled D₆-ADGV, D₆-SDGV were custom synthesized by PepTech (Saint Petersburg, Russia) with purity over 96%. TDCA and ammonium formate were purchased from Merck & Co. (Darmstadt, Germany). The deuterated form of D₄-TDCA was obtained from Cayman Chemical (MI, USA). Acetonitrile (ACN), iso-propanol (IPA), Methanol (MeOH), formic acid (FA), and ultra-pure water were LCMS grade purchased from Thermo Fisher Scientific (Langenselbold, Germany).

### Participants and Samples

544 subjects had liver NMR performed, the most accurate method for determining liver fat content. Blood was collected in EDTA-coated vacutainers.

The whole blood was immediately centrifuged at 2,000 g for 10 min at 10° C. The supernatant, designated plasma was carefully transferred to an Eppendorf tube and stored at −80° C. till further analysis.

### Preparation of Standards Solutions

Calibrations standards were prepared in mobile phase A at concentrations ranging from 0 ng/mL to 200 ng/mL for ADGV and SDGV, from 0 to 500 ng/mL for TDCA. Deuterated standards of D₆-ADGV, D₆-SDGV as internal standards were added to all calibration standards at concentrations of 5 ng/mL, and D₄-TDCA at 10 ng/mL, respectively.

### Plasma Sample Preparation

Aliquots of 10 μL plasma were transferred to 96-well collection plate (Biotage AB, Uppsala, Sweden) and extracted with 100 μL of extraction solvents (ACN: MeOH: FA, 75%: 25%:0.2%, vol:vol:vol) containing 5 ng/mL D₆-ADGV, D₆-SDGV, and 10 ng/mL D₄-TDCA, respectively. The mixture was vortexed for 30 s before being incubated on ice for 30 min for protein precipitation, followed by centrifugation at 3,900 rpm for 25 min at 4° C. using a benchtop centrifuge (Eppendorf 8010R, Hamburg, Germany). The supernatant was collected and transferred to Eppendorf tubes before drying in a Speedvac vacuum concentrator with refrigerated vapor trap (Thermo Fisher Scientific). The dried samples were reconstituted with 100 μL of mobile phase A and centrifuged at 15,000 rpm at 4° C. for 15 min to remove any potential particulates presented. The resulting supernatant of 70 μL was carefully transferred to a HPLC insert and submitted for LC-MS/MS analysis.

### LC-MS/MS Conditions

The Liquid chromatography tandem mass spectrometry system (LCMS) was composed of a Shimadzu Nexera LC-40 series coupled with a triple quadrupole mass spectrometer 8050 (Shimadzu Co., Kyoto, Japan). The chromatographic separations were achieved on a Hypercarb PGC column (50×2.1 mm, 5 μm, Thermo Fisher Scientific) equipped with a guard column (HyperSep, 10×2.1 mm, 5 μm) using mobile phase A consisted of 0.1% FA and 10 mM ammonium formate in water (pH=3.6) and mobile phase B containing 0.1% FA, 10 mM ammonium formate in 90% IPA and 10% ACN. The gradient was started at 0% B and ramped to 8% at 9 min with flow rate at 0.6 mL/min, and then increased rapidly to 98% within 1 min and maintained for another 9 min with flow rate at 0.8 mL/min before returning to 0% B at 20 mM. The equilibration time was 4 min to ensure minimal carryover.

The mass spectrometry was operated with an electrospray ionization (ESI) source in positive mode. Nitrogen was used as nebulizing gas with flow set at 3 I/min, drying gas and heating gas flows were maintained at 10 I/min. The interface voltage was 3.5 KV. The desolvation line temperature, block heater and interface temperatures were optimized to 250° C., 400° C. and 300° C., respectively. High purity argon (99.99%) was used as collision gas at 270 kPa.

TABLE 8
The multiple reaction monitoring (MRM) transitions were optimized by
direct flow injection of 0.5 μL of standards at 1 μg/mL.

| | MRM | | | |
|---|---|---|---|---|
| | transitions | Q1 Ore- | Collision | Q3 Pre- |
| Compound | (m/z) | Bias (V) | Energy | Bias (V) |
| ADGV | 202.1 -> 71.1 | −26.8 | −25.6 | −26.8 |
| | 202.1 -> 70.1 | −23.7 | −24 | −26.8 |
| | 202.1 -> 46.1 | −20.6 | −22.5 | −26.8 |
| D₆-ADGV | 208.2 -> 77.1 | −25.8 | −26.1 | −25.8 |
| | 208.2 -> 70.1 | −25.8 | −24.5 | −25.8 |
| | 208.2 -> 52.1 | −19.4 | −26.1 | −25.8 |
| SDGV | 202.1 -> 71.1 | −25.8 | −22.9 | −25.8 |
| | 202.1 -> 70.1 | −25.8 | −16.5 | −25.8 |
| | 202.1 -> 88.1 | −19.4 | −14.2 | −25.8 |
| D₆-SDGV | 208.25 -> 77.15 | −25.8 | −27.8 | −25.8 |
| | 208.25 -> 70.1 | −21 | −22.3 | −25.8 |
| | 208.25 -> 71.15 | −12.9 | −18.4 | −25.8 |
| D₄-TDCA | 504.15 -> 468.35 | −14.5 | −17.4 | −32.3 |
| | 504.15 -> 126.25 | −14.5 | −36.8 | −48.4 |
| TDCA | 500.4 -> 464 | −15.2 | −18.2 | −33.2 |
| | 500.4 -> 122.2 | −14.5 | −36.8 | −48.8 |

### Results

### Separation of Isomers and TDCA

Isomers of ADGV and SDGV that share the same molecular weight (201.2 g/mol) were successfully separated on the hypercarb column as shown in FIG. 6A. Identities of both isomers were confirmed by injecting either ADGV or SDGV alone to obtain individual retention times. ADGV was eluted slightly earlier (0.4-0.5 min) than SDGV when injecting the individual standard. During MS/MS scan, both isomers have the same most prominent product ions, except difference on one MRM transition (Table 8), that is 202.1->46.1 for ADGV and 202.1->52.1 for SDGV. The deuterated forms of ADGV and SDGV were demonstrated the same fragment patterns, with additional 6 AMU heavier than their native forms (Table 8). When detecting both isomers in biological samples, there were two peaks eluted for each compound (FIG. 6B) noting the common MRM transitions in MS/MS monitoring for both compounds. Integrated areas were obtained from the earlier eluting peak (5.2 min) for ADGV, and the later peak (5.7 min) for SDGV, respectively. The same practice was applied for the internal standard values of deuterated forms of ADGV and SDGV.

TDCA is a primary bile acid that is hydrophobic compared to ADGV and SDGV. A high percentage (98%) of mobile phase B was used to be able to elute TDCA on the same hypercarb column. At 14.3 min, TDCA and its deuterated form of D₄-TDCA were detected as shown in FIG. 6C.

### Standard Curves and Assay Validation

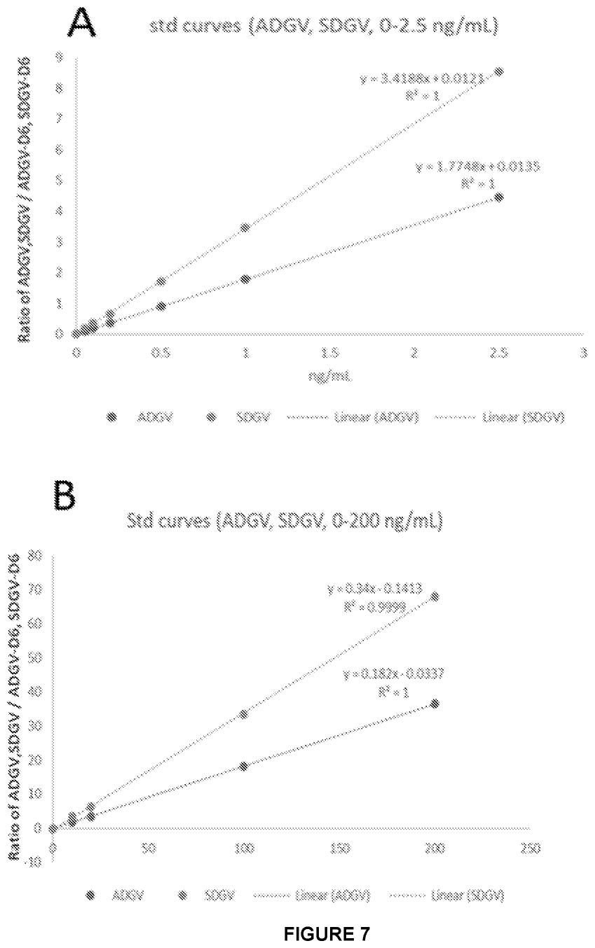

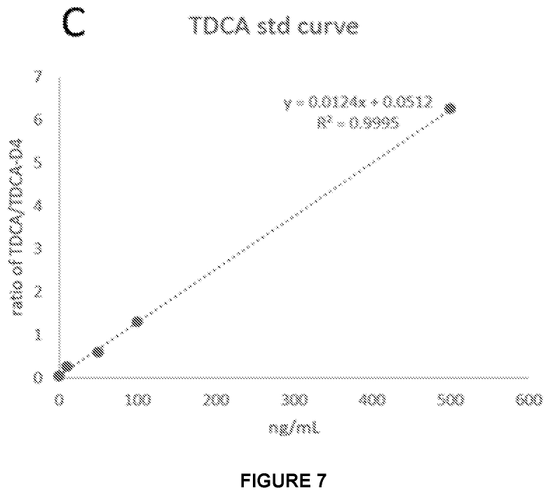

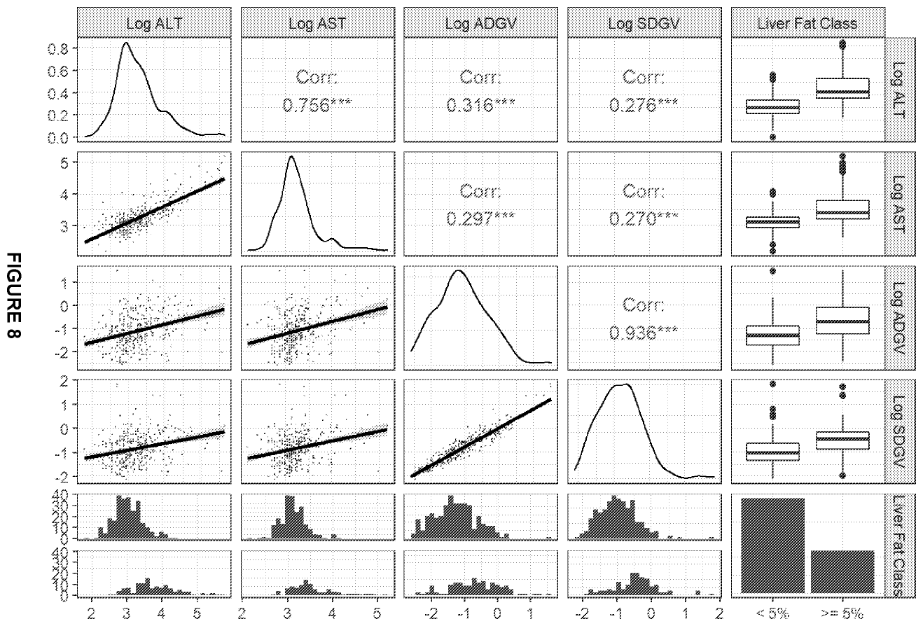

Standard curves of three compounds (ADGV, ADGV and TDCA) showed excellent linear correlations from 0 ng/mL to 200 ng/mL for ADGV, SDGV, and 0-500 ng/mL for TDCA, respectively (FIG. 7). Excellent reproducibility was observed in all three standard curves with no weighing method necessary. The low detection limit in the current method settings was 0.5-1 ng/mL for TDCA and lower femtogram (between 2.5-5 fg/mL) for ADGV and SDGV, respectively (data not shown). This accuracy of this assay was assessed by analysing three different concentrations of each compound in pooled human plasma. The pooled plasma without addition of any standard was used as QC-zero, spiked 50 ng/mL of each standard into pooled plasma as QC-medium, and spiked 100 ng/mL of each standard into pooled plasma as QC-high. Each compound was analysed 15-17 times over two weeks and the resulting peak areas were calculated using the standard curves to obtain the final concentrations. Relative Standard Deviations (RSDs) were calculated using final concentrations with acceptance criterion as ±15%. Specifically, RSDs were 7.03% and 7.44% for ADGV and SDGV, respectively, while RSD of 13.82% was obtained for TDCA at quality control samples.

### Simultaneous Quantifications of SDGV and TDCA on Finland Cohort

With a total of 544 plasma samples, four sets of standard curves were incorporated into the whole worklist and distributed in the first, second, third quarters and last quarter of the worklist to mitigate any signal deviations. One blank sample (mobile phase A), and duplicate QC samples with zero standard spiking, medium concentration spike and high concentration spike were placed in every 20 samples to evaluate the reproducibility, accuracy, and robustness of the assay. Procedure blanks (mobile phase A was extracted with extraction solvents) were also analysed to monitor any cross contaminations during plasma sample preparation.

As shown below, SDGV was found a more sensitive biomarker than ADGV to reflect and track changes of fatty liver. In order to sensitively and accurately determine SDGV and TDCA, levels of SDGV and TDCA were quantified by monitoring ratios of SDGV and TDCA over internal standard D₆-SDGV and D₄-TDCA, respectively. The final absolute concentrations were calculated from the standard curves.

### Calculation of Normal Upper Normal Levels of SDGV Using 5% Liver Fat Threshold.

Recent determination of liver fat with optimised methods, such as MR spectroscopy, have determined that up to 5% liver fat is normal. Commonly, ultrasound is used these days to detect liver fat, but is only determined at the 20% liver fat level.

Therefore, in order to determine the normative upper limit of normal (ULN) for SDGV, we used the 5% level of liver fat. At this level, 0.516 uM was the ULN using the Youden Index (maximisation of sensitivity and specificity). This is the first report of ULN for SDGV in a population at the appropriate threshold of liver fat (5%).

### Replication of TDCA Detection of HCC in NAFLD in Two Cohorts

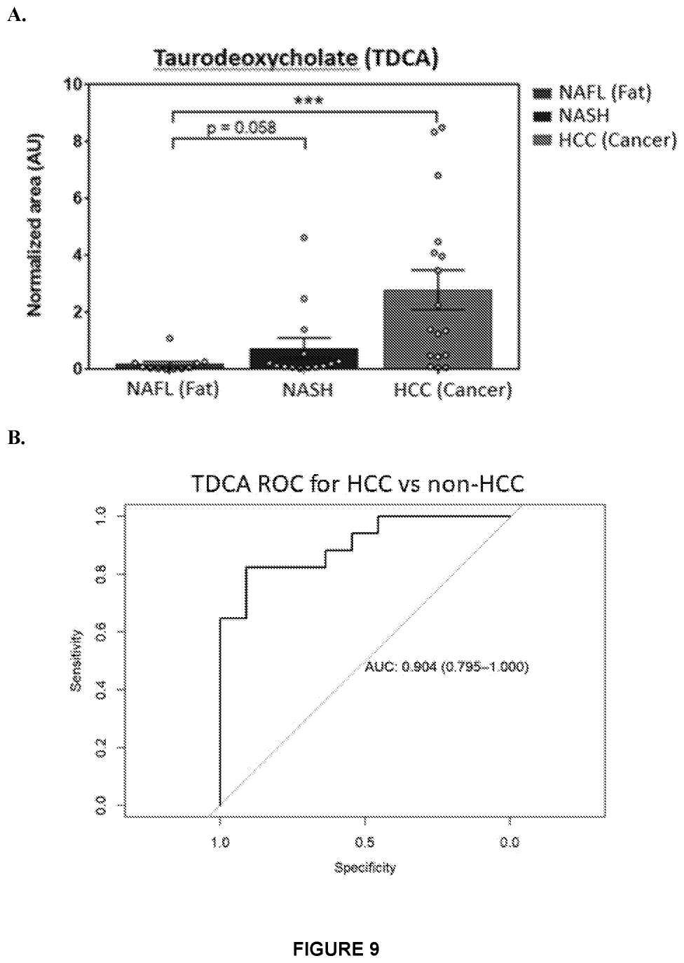

It was determined that TDCA is able to distinguish HCC from non-HCC within NAFLD patients (FIGS. 9A and 9B) in a cohort from the USA. The ROC demonstrated high diagnostic performance of TDCA with a C-statistic of 0.9.

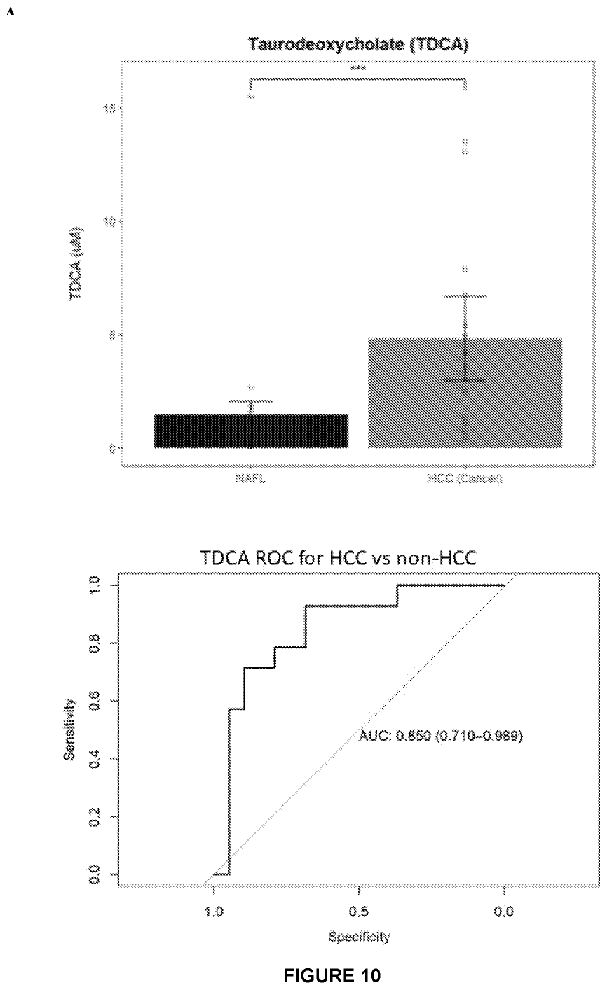

This result was confirmed in a cohort of patients from Finland (FIGS. 10A and 10B). The ROC again demonstrated high diagnostic accuracy with a C-statistic of 0.85.

The optimal cut point for TDCA distinguishing between HCC vs non-HCC was 0.715 uM. As a paired diagnostic, the present inventors propose that SDGV is the best available plasma biomarker for identifying NAFLD. Within this group, TDCA is the only plasma biomarker capable of distinguishing HCC from non-HCC.

Based on the above, a paired diagnostic approach was conceived as below:

| | |
|---|---|
| SDGV < 0.5 μM / | Patient is unlikely to have fatty |
| TDCA < 0.7 μM → | liver disease (recommend eat |
| | healthily & exercise, retest in 2 |
| | years if still overweight |
| SDGV > 0.5 μM / | Patient is likely to have fatty |
| TDCA < 0.7 μM → | liver disease and be on the |
| | pathway to diabetes and heart |
| | disease (recommend to consult |
| | with physician on lifestyle |
| | measures including eating |
| | healthily & exercising to promote |
| | weight loss. Response to be |
| | tracked by measuring SDGV |
| | levels. |
| SDGV > 0.5 μM / | Imaging of liver urgently |
| TDCA > 0.7 μM → | recommended as patient may have |
| | significant fatty liver diseases |

### Discussion

In addition to demonstrating the superior diagnostic performance of SDGV compared to all other available plasma biomarkers, the present inventors have determined the normative upper limit of normal (ULN) for SDGV sing the most sensitive imaging available for detection of liver fat. At the 5% level of liver fat, the ULN for SDGV was 0.516 uM. This represents the first report of SDGV ULN in a human population whereby liver fat levels were determined at the normal levels of liver fat, an important determinant of circulating SDGV levels.

The present inventors then proceeded to interrogate the ability of TDCA to distinguish NAFLD-derived HCC from non-HCC. Remarkably, TDCA showed similar diagnostic performance in two unrelated cohorts, one from USA and one from Finland. The ROC C-statistics showed excellent discrimination, 0.9 in the USA cohort and 0.85 in the Finnish cohort. The present inventors then determined the optimal cut point for distinguishing HCC from non-HCC within an NAFLD cohort, being 0.715 uM.

Whilst detecting and tracking liver fat is important, and SDGV demonstrated superior performance in both, in most cases NAFLD has good outcomes. It is the small number of cases that progress that require urgent attention. Being able to detect those on course for progression and the worst possible outcome, i.e. HCC, is of utmost importance. Reassurance and avoidance of unnecessary and expensive further investigation would result from a reliable and accurate diagnostic of HCC in this context. Therefore, TDCA is a highly valuable diagnostic that can prioritise investigations in those who need it and prevent healthcare systems being overwhelmed with unnecessary investigations in the vast majority of cases of a highly prevalent disease (1 in 4 adults).

### REFERENCES

1 Foster, K. J., Dewbury, K. C., Griffith, A. H. & Wright, R. The accuracy of ultrasound in the detection of fatty infiltration of the liver. Br J Radiol 53, 440-442, doi:10.1259/0007-1285-53-629-440 (1980).
2 Joseph, A. E., Saverymuttu, S. H., al-Sam, S., Cook, M. G. & Maxwell, J. D. Comparison of liver histology with ultrasonography in assessing diffuse parenchymal liver disease. C/in Radiol 43, 26-31, doi:10.1016/s0009-9260(05)80350-2 (1991).
3 Needleman, L. et al. Sonography of diffuse benign liver disease: accuracy of pattern recognition and grading. AJR Am J Roentgenol 146, 1011-1015, doi:10.2214/ajr.146.5.1011 (1986).
4 Saverymuttu, S. H., Joseph, A. E. & Maxwell, J. D. Ultrasound scanning in the detection of hepatic fibrosis and steatosis. Br Med J (C/in Res Ed) 292, 13-15, doi:10.1136/bmj.292.6512.13 (1986).
5 Mottin, C. C. et al. The role of ultrasound in the diagnosis of hepatic steatosis in morbidly obese patients. Obes Surg 14, 635-637, doi:10.1381/096089204323093408 (2004).
6 Park, S. H. et al. Macrovesicular hepatic steatosis in living liver donors: use of CT for quantitative and qualitative assessment. Radiology 239, 105-112, doi:10.1148/radiol.2391050361 (2006).
7 Kodama, Y. et al. Comparison of CT methods for determining the fat content of the liver. AJR Am J Roentgenol 188, 1307-1312, doi: 10.2214/AJR.06.0992 (2007).
8 Bohte, A. E., van Werven, J. R., Bipat, S. & Stoker, J. The diagnostic accuracy of US, CT, MRI and 1H-MRS for the evaluation of hepatic steatosis compared with liver biopsy: a meta-analysis. Eur Radiol 21, 87-97, doi:10.1007/s00330-010-1905-5 (2011).
9 Szczepaniak, L. S. et al. Measurement of intracellular triglyceride stores by H spectroscopy: validation in vivo. Am J Physiol 276, E977-989, doi:10.1152/ajpendo.1999.276.5.E977 (1999).
10 Szczepaniak, L. S. et al. Magnetic resonance spectroscopy to measure hepatic triglyceride content: prevalence of hepatic steatosis in the general population. Am J Physiol Endocrinol Metab 288, E462-468, doi:10.1152/ajpendo.00064.2004 (2005).
11 O'Sullivan, J. F. et al. Dimethylguanidino valeric acid is a marker of liver fat and predicts diabetes. J Clin Invest 127, 4394-4402, doi:10.1172/JCI95995 (2017).
12 Lin, S. et al. Comparison of MAFLD and NAFLD diagnostic criteria in real world. Liver Int 40, 2082-2089, doi:10.1111/liv.14548 (2020).
13 Valenti, L. & Pelusi, S. Redefining fatty liver disease classification in 2020. Liver Int 40, 1016-1017, doi:10.1111/liv.14430 (2020).
14 Andreas Fritsche, R. W., Martin Heni, Kostantinos Kantartzis, Jurgen Machann, Fritz Schick, Rainer Lehmann, Andreas Peter, Corinna Dannecker, Louise Fritsche, Vera Valenta, Renate Schick, Peter Paul Nawroth, Stefan Kopf, Andreas FH Pfeiffer, Stefan Kabisch, Ulrike Dambeck, Michael Stumvoll, Matthias BliTher, Andreas L Birkenfeld, Peter Schwarz, Hans Hauner, Julia Clavel, Jochen SeiBler, Andreas Lechner, Karsten Müssig, Katharina Weber, Michael Laxy, Stefan Bornstein, Annette Schiirmann, Michael Roden, Martin Hrabe de Angelis, Norbert Stefan, Hans-Ulrich Haring. Risk-stratified lifestyle intervention to prevent type 2 diabetes. medRxiv, doi:https://doi.org/10.1101/2021.01.26.21249582 (2021).
15 Martens-Lobenhoffer, J. & Bode-Boger, S. M. Quantification of L-arginine, asymmetric dimethylarginine and symmetric dimethylarginine in human plasma: a step improvement in precision by stable isotope dilution mass spectrometry. J Chromatogr B Analyt Technol Biomed Life Sci 904, 140-143, doi:10.1016/j.jchromb.2012.07.021 (2012).
16 μMartens-Lobenhoffer, J., Surdacki, A., Bode-Boger S. M. Fast and precise quantification of L-homoarginine in human plasma by HILIC-isotope dilution-MS-MS. Chromatographia 76, 1755-1759, doi:10.1007/s10337-013-2561-0 (2013).
17 Jarzebska, N. et al. Kidney and liver are the main organs of expression of a key metabolic enzyme alanine:glyoxylate aminotransferase 2 in humans. Atheroscler Suppl 40, 106-112, doi:10.1016/j.atherosclerosissup.2019.08.041 (2019).
18 μMartens-Lobenhoffer, J., Rodionov, R. N. & Bode-Boger, S. M. Probing AGXT2 enzyme activity in mouse tissue by applying stable isotope-labeled asymmetric dimethyl arginine as substrate. J Mass Spectrom 47, 1594-1600, doi:10.1002/jms.3125 (2012).
19 Bedogni, G. et al. The Fatty Liver Index: a simple and accurate predictor of hepatic steatosis in the general population. BMC Gastroenterol 6, 33, doi:10.1186/1471-230X-6-33 (2006).
20 Gastaldelli, A. et al. Fatty liver is associated with insulin resistance, risk of coronary heart disease, and early atherosclerosis in a large European population. Hepatology 49, 1537-1544, doi:10.1002/hep.22845 (2009).
21 Shen, Y. N. et al. External validation of non-invasive prediction models for identifying ultrasonography-diagnosed fatty liver disease in a Chinese population. Medicine (Baltimore) 96, e7610, doi: 10.1097/MD.0000000000007610 (2017).
22 Fedchuk, L. et al. Performance and limitations of steatosis biomarkers in patients with nonalcoholic fatty liver disease. Aliment Pharmacol Ther 40, 1209-1222, doi:10.1111/apt.12963 (2014).
23 Rodionov, R. N., Jarzebska, N., Weiss, N. & Lentz, S. R. AGXT2: a promiscuous aminotransferase. Trends Pharmacol Sci 35, 575-582, doi:10.1016/j.tips.2014.09.005 (2014).
24 Ogawa, T., Kimoto, M. & Sasaoka, K. Purification and properties of a new enzyme, NG,NG-dimethylarginine dimethylaminohydrolase, from rat kidney. J Biol Chem 264, 10205-10209 (1989).

1. (canceled)

2. A method for determining a presence or absence of liver fat in a subject, or determining a subject's level of liver fat, the method comprising measuring the level of symmetric dimethylguanidino valeric acid (SDGV) in a biological sample obtained from the subject, and determining said presence or absence or level of liver fat based on the level of SDGV in the biological sample.

3. (canceled)

4. The method according to claim 2 further comprising determining a change in liver fat level in a subject, comprising measuring the level of symmetric dimethylguanidino valeric acid (SDGV) in one or more biological samples obtained from the subject at multiple timepoints, and determining whether there is a change in liver fat level in the subject over time based on a comparison of the level of SDGV in each said biological sample.

5. The method of claim 2, wherein the subject has a fatty liver disease (FLD).

6. A method for diagnosing and/or prognosing fatty liver disease (FLD) in a subject, the method comprising measuring the level of symmetric dimethylguanidino valeric acid (SDGV) in a biological sample obtained from the subject, and determining whether the subject has fatty liver disease based on the level of SDGV in the biological sample.

7. The method of claim 6, wherein the fatty liver disease (FLD) is non-alcoholic fatty liver disease (NAFLD).

8. The method of claim 2, wherein measuring the level of symmetric dimethylguanidino valeric acid (SDGV) in the biological sample is performed without measuring the level of asymmetric dimethylguanidino valeric acid (ADGV) in the biological sample; and/or
wherein said determining based upon the level of SDGV in the biological sample is performed without determining based on the level of asymmetric dimethylguanidino valeric acid (ADGV) in the biological sample.

9. (canceled)

10. The method of claim 2, wherein the level of symmetric dimethylguanidino valeric acid (SDGV) in the biological sample is compared to that of a SDGV control.

11. The method of claim 2, wherein:
said measuring further comprises measuring the level of a further biological marker in the biological sample selected from any one or more of: alanine transaminase (ALT), aspartate transaminase (AST), alkaline phosphatase (ALP), albumin, albumin and total protein, bilirubin, gamma-glutamyltransferase (GGT), L-lactate dehydrogenase (LD), prothrombin time (PT) and/or taurodeoxycholic acid (TDCA); and
said determining is additionally based on the level of the further biological marker in the biological sample,
optionally wherein the level of the further biological marker in the biological sample is compared to that of a further control.

12. (canceled)

13. The method of claim 10, wherein the SDGV control is selected from any one or more of:
a biological sample from a subject without liver fat, or without clinically acceptable levels of liver fat;
a series of biological samples from subjects without liver fat or without clinically acceptable levels of liver fat;
a standard control value or a set of standard control values indicative of the presence or absence of liver fat.

14. The method of claim 13, wherein the clinically acceptable levels of liver fat are each: less than 20% fat content in liver by weight; less than 15% fat content in liver by weight; less than 10% fat content in liver by weight; less than 8% fat content in liver by weight; less than 7% fat content in liver by weight; less than 6% fat content in liver by weight; less than 5% fat content in liver by weight; or less than 4% fat content in liver by weight;
wherein the standard control value or a set of standard control values indicative of the presence of liver fat is/are each: more than 4% fat content in liver by weight; more than 5% fat content in liver by weight; more than 6% fat content in liver by weight; more than 7% fat content in liver by weight; more than 8% fat content in liver by weight; more than 10% fat content in liver by weight; more than 15% fat content in liver by weight; or more than 20% fat content in liver by weight; and/or
wherein the standard control value or a set of standard control values indicative of the absence of liver fat is/are each: less than 20% fat content in liver by weight; less than 15% fat content in liver by weight; less than 10% fat content in liver by weight; less than 8% fat content in liver by weight; less than 7% fat content in liver by weight; less than 6% fat content in liver by weight; less than 5% fat content in liver by weight; or less than 4% fat content in liver by weight.

15. (canceled)

16. (canceled)

17. The method of claim 2, wherein the biological sample is a blood, serum, plasma, urine or liver sample; optionally
further comprising culturing the biological sample prior to said determining; and/or
comprising the step of obtaining the biological sample from the subject.

18.-20. (canceled)

21. The method of claim 2, wherein the measuring of the level of symmetric dimethylguanidino valeric acid (SDGV) in the biological sample is conducted by any one or more of: tandem liquid chromatography-mass spectrometry (LC-MS/MS), gas chromatography-mass spectrometry (GC-MS/MS), and/or nuclear magnetic resonance spectroscopy (NMR).

22. A method for diagnosing and/or prognosing hepatocellular carcinoma (HCC) in a subject, the method comprising:
determining if the subject has non-alcoholic fatty liver disease (NAFLD)_by measuring the level of symmetric dimethylguanidino valeric acid (SDGV) in a biological sample obtained from the subject, and determining whether the subject has NAFLD disease based on the level of SDGV in the biological sample; and
determining whether the subject has HCC by measuring the level of taurodeoxycholic acid (TDCA) in a biological sample obtained from the subject, and determining whether the subject has HCC based on the level of TDCA in the biological sample.

23. (canceled)

24. (canceled)

25. The method of claim 22, wherein:
the level of symmetric dimethylguanidino valeric acid (SDGV) in the biological sample is compared to that of a SDGV control; and/or
the level of the TDCA in the biological sample is compared to that of a TDCA control.

26. The method of claim 25, wherein the SDGV control and/or the TDCA control is selected from any one or more of:
a biological sample from a subject without liver fat and without HCC, or without clinically acceptable levels of liver fat and without HCC;
a series of biological samples from subjects without liver fat and without HCC, or without clinically acceptable levels of liver fat and without HCC;
a standard control value or a set of standard control values indicative of the presence or absence of liver fat and/or HCC.

27. The method of claim 22, wherein said determining if the subject has non-alcoholic fatty liver disease (NAFLD) comprises determining a concentration of the SDGV in plasma from the subject of above 0.4 μM, above 0.45 μM, above 0.5 μM, above 0.51 μM, above 0.516 μM, above 0.52 μM or above 0.525 μM, to thereby determine that the subject has NAFLD; and/or
wherein said determining whether the subject has HCC comprises determining a concentration of the TDCA in a plasma from the subject of above, above 0.7 μM, above 0.75 μM, or above 0.8 μM, to thereby determine that the subject has HCC.

28. (canceled)

29. The method of claim 22, comprising:
determining a concentration of the SDGV in plasma from the subject of below 0.525 μM, below 0.52 μM, below 0.516 μM, below 0.51 μM, below 0.5 μM, below 0.45 μM or below 0.4 μM; and/or
determining a concentration of the TDCA in a plasma from the subject of below 0.8 μM, below 0.75 μM, or below 0.7 μM;
to thereby determine that the subject does not have HCC.

30. The method of claim 22, comprising:
determining a concentration of the SDGV in plasma from the subject of above 0.4 μM, above 0.45 μM, above 0.5 μM, above 0.51 μM, above 0.516 μM, above 0.52 μM or above 0.525 μM, to thereby determine that the subject has NAFLD; and
determining a concentration of the TDCA in a plasma from the subject of below 0.8 μM, below 0.75 μM, or below 0.7 μM;
to thereby determine that the subject should be monitored due to a risk of developing HCC.

31. The method of claim 30, wherein the subject is monitored by periodic measurement of SDGV levels in plasma from the subject and/or periodic measurement of TDCA levels in plasma from the subject, and/or wherein the method further comprises either of:
(i) treating the subject to reduce liver fat in the subject;
(ii) treating the subject to alleviate a disease arising at least in part from or characterised by excess liver fat.

32. (canceled)

33. (canceled)

34. The method of claim 22, further comprising either of:
(i) treating the subject to reduce liver fat in the subject;
(ii) treating the subject to alleviate a disease arising at least in part from or characterised by excess liver fat.

35.-41. (canceled)
